(19) United States
(12) Patent Application Publication
(10) Pub. No.: US 2021/0077547 A1
(43) Pub. Date: Mar. 18, 2021

(54) SPORE-BASED PROBIOTIC COMPOSITION FOR MODULATION OF MICROBIOME IN HUMANS

(71) Applicant: Microbiome Labs, LLC, Saint Augustine, FL (US)

(72) Inventors: Kiran Krishnan, Saint Augustine, FL (US); Dale M. Kriz, Saint Augustine, FL (US); Thomas F. Bayne, Saint Augustine, FL (US)

(73) Assignee: Microbiome Labs, LLC, Saint Augustine, FL (US)

(21) Appl. No.: 17/020,610

(22) Filed: Sep. 14, 2020

Related U.S. Application Data
(60) Provisional application No. 62/899,301, filed on Sep. 12, 2019.

(51) Int. Cl. A61K 35/745; A61K 35/742; A61K 35/741; A61P 1/14

(52) U.S. Cl. CPC A61K 35/745; A61P 1/14; A61K 35/741; A61K 35/742

(57) ABSTRACT

A spore-based probiotic composition is provided that comprises at least one viable probiotic microorganism having a biological or therapeutic effect on microbiome in humans. One exemplary composition contains five different strains of Bacillus spp. Also provided are methods of producing spore-based probiotic compositions. A validated in vitro gut model which is a simulated human intestinal microbial ecosystem reactor was used to assess the long-term effect of the composition on microbial metabolic activity and community composition. The results support use of the composition in protecting against obesity-related disorders, metabolic disorders, inflammation, and cancer, for example. A method for modulating microbial metabolic activity and/or modulating microbial community composition in a human subject is described.

This application claims the benefit of U.S. Provisional application No. 62/899,301, filed on Sep. 12, 2019, which is hereby incorporated by reference herein in its entirety.

### TECHNICAL FIELD

The present disclosure relates to the field of spore-based probiotic compositions. A spore-based probiotic composition is provided that comprises at least one viable probiotic microorganism having a biological or therapeutic on microbiome in humans. One exemplary composition contains five different strains of Bacillus spp. Also provided are methods of producing spore-based probiotic compositions.

### BACKGROUND

The microbiome is the genetic material of all microbes (bacteria, fungi, protozoa, and viruses) that live on or in the human body. Microbes outnumber human cells in a 10:1 ratio. Most microbes live in the gut, particularly the large intestine. The number of genes of all microbes in the microbiome is 200-fold that of the human genome. The microbiome may weigh as much as 2 kg. The bacteria help digest food, regulate the immune system, protect against other bacteria that cause disease, and produce vitamins (including the B vitamins B12, thiamine, and riboflavin; and Vitamin K, which is required for blood coagulation). The microbiome became generally recognized in the late 1990s. See, e.g., Marilyn Hair & Jon Sharpe, Fast facts about the human microbiome, CTR. FOR ECOGENETICS & ENVTL. HEALTH, UNIV. WASHINGTON (2014), incorporated by reference herein in its entirety.

The microbiome is essential for human development, immunity, and nutrition. Bacteria living in and on humans are not invaders but, rather, beneficial colonizers. Autoimmune diseases including diabetes, rheumatoid arthritis, muscular dystrophy, multiple sclerosis, and fibromyalgia are associated with dysfunctional microbiomes. Disease-causing microbes accumulate over time and change genetic activities and metabolic processes, triggering abnormal immune responses against substances and tissues that are, in fact, part of a healthy body. Autoimmune diseases appear to run in families not because of germline inheritance but, rather, by inheritance of the familial microbiome. See, e.g., Hair & Sharpe, 2014.

Humans are essentially sterile during gestation. During and after birth, however, every bodily surface, including the skin, mouth, and gut, becomes host to an enormous variety of microbes: bacterial, archaeal, fungal, and viral. Under normal circumstances, the microbes aid in food digestion and maintenance of immune systems; dysfunctional human microbiotas have been linked to conditions ranging from inflammatory bowel disease to antibiotic-resistant infections. See, e.g., X. C. Morgan & C. Huttenhower, Chapter 12: human microbiome analysis, 8 PLoS COMPUTATIONAL BIOLOGY e1002808 (2012), incorporated by reference herein in its entirety.

The gut microbiota is essential to human health throughout life. The gut microbiome is a vast collection of bacteria, viruses, fungi, and protozoa that colonize the gastrointestinal tract and outnumber human cells 10-fold. Exposures in early life [Mode of delivery (maternal microbes); infant diet (selective substrates); antibiotics (selective killing); probiotics (selective enrichment); and physical environment (environmental microbes)] results in colonization of gut microbiota which contributes to the development of the immune system, intestinal homeostasis and host metabolism. Disruption of the gut microbiota is associated with a growing number of diseases. See, e.g., M. B. Azad, et al., Gut microbiota of healthy Canadian infants: profiles by mode of delivery and infant diet at 4 months, 185 CAN. MED. ASS'N J. 385 (2013), incorporated by reference herein in its entirety. Recent advances in metagenomics have enhanced our understanding of the gut microbiome, suggesting that it can provide important immune and metabolic benefits to humans.

Interestingly, the intestinal microbiota affects the immune and/or inflammatory status of the host by modulating intestinal barrier function and by influencing the development of the immune response. The gut microbiome's influence on the human immune system is far-reaching and intricately designed to enable immune tolerance of dietary and environmental antigens and provide protection against potential pathogens and toxins. Several gut microbial structures that play an important role in barrier functions have been identified. The secreted protein, p40, from Lactobacilli LGG ameliorates cytokine-mediated apoptosis and disruption of the gut epithelial barrier, and flagellin from Escherichia coli Nissle is associated with induction of β-defensin 2 in epithelial cells. See, e.g., F. Yan, et al., Colon-specific delivery of a probiotic-derived soluble protein ameliorates intestinal inflammation in mice through an EGFR-dependent mechanism, 121 J. CLINICAL INVESTIGATION 2242 (2011); M. Schlee, et al., Induction of human beta-defensin 2 by the probiotic Escherichia coli Nissle 1917 is mediated through flagellin, 75 INFECTION & IMMUNITY 2399 (2007); each of which is incorporated by reference herein in its entirety. Gut microbiota has been shown to direct maturation of the host immune system, to play a key role in the induction of immunoglobulin (“Ig”) A and germinal centers, and to drive Th1, Th17, and regulatory T cell (“Treg”) development in the gut. See, e.g., S. K. Mazmanian, et al., An immunomodulatory molecule of symbiotic bacteria directs maturation of the host immune system, 122 CELL 107 (2005); H. L. Klaasen, et al., Intestinal, segmented, filamentous bacteria in a wide range of vertebrate species, 27 LABANIMAL 141 (1993); G. L. Talham, et al., Segmented filamentous bacteria are potent stimuli of a physiologically normal state of the murine gut mucosal immune system, 67 INFECTION & IMMUNITY 1992 (1999); H. Bauer, et al., The response of the lymphatic tissue to the microbial flora. Studies on germfree mice, 42 AM. J. PATHOLOGY 471 (1963); K. Atarashi, et al., Induction of colonic regulatory T cells by indigenous Clostridium species, 331 SCIENCE 337 (2011); V. Gaboriau-Routhiau, et al., The key role of segmented filamentous bacteria in the coordinated maturation of gut helper T cell responses, 31 IMMUNITY 677 (2009); I. I. Ivanov, et al., Induction of intestinal Th17 cells by segmented filamentous bacteria, 139 CELL 485 (2009); each of which is incorporated by reference herein in its entirety. In most individuals, the commensal-mediated induction of these different components of the immune response is beneficial for host health. However, the composition of the gut microbiota can differentially influence various immune cell populations and adversely affect autoimmune/inflammatory disease-susceptible hosts, e.g., the presence of segmented filamentous bacteria (“SFB”) has been associated with a strong Th17 response and development of Th17-mediated diseases. See, e.g., Y. K. Lee, et al., Proinflammatory T-cell responses to gut microbiota promote experimental autoimmune encephalomyelitis, 108 (Suppl. 1) PROCEEDINGS NAT'L ACAD. SCI. USA 4615 (2011); R. Stepankova, et al., Segmented filamentous bacteria in a defined bacterial cocktail induce intestinal inflammation in SCID mice reconstituted with CD45RBhigh CD4+ T cells, 13 INFLAMMATORY BOWEL DISEASES 1202 (2007); H. J. Wu, et al., Gut-residing segmented filamentous bacteria drive autoimmune arthritis via T helper 17 cells, 32 IMMUNITY 815 (2010); each of which incorporated by reference herein in its entirety.

Interestingly, the gut microbiome and skin are uniquely connected in purpose and function. As the primary interface with the external environment, both organs are crucial in maintaining overall homeostasis. Recent research has demonstrated a strong bidirectional connection between the gut and skin, suggesting that digestive health plays a pivotal role in skin homeostasis and allostasis. Gut bacteria have been shown to participate in the pathophysiology of many inflammatory disorders, including skin disorders such as acne, atopic dermatitis (“AD”), scleroderma, vitiligo, rosacea, and psoriasis.

Skin and mucosal surfaces of mammalian species are populated by millions of bacteria that impart diverse metabolic effects. See, e.g., J. K. Nicolson, et al., Host-gut microbiota metabolic interactions, 336 SCIENCE 1262 (2012), incorporated by reference herein in its entirety. These host-associated microbes play a well-established role in homeostasis in the gastrointestinal (“GI”) tract. See, e.g., Y. K. Lee & S. K. Mazmanian, Has the microbiota played a critical role in the evolution of the adaptive immune system?, 330 SCIENCE 1768 (2010); N. P. McNulty, et al., The impact of a consortium of fermented milk strains on the gut microbiome of gnotobiotic mice and monozygotic twins, 3 SCI. TRANSLATIONAL MEDICINE 106ra106; each of which is incorporated by reference herein in its entirety. There is now substantial evidence linking various gut microbiota and local immunity networks with systematic effects on the immune system. See, e.g., T. Chinen & A. Y. Rudensky, The effects of commensal microbiota on immune cell subsets and inflammatory responses, 245 IMMUNOLOGICAL REVIEWS 45 (2012); L. V. Hooper, et al., Interactions between the microbiota and the immune system, 336 SCIENCE 1268 (2012); C. L. Maynard, et al., Reciprocal interactions of the intestinal microbiota and immune system, 489 NATURE 231 (2012); each of which is incorporated by reference herein in its entirety. Disruption of the normal balance between microbial communities in the intestine is associated with allergic, autoimmune, metabolic, and neoplastic pathologies in the GI tract and other distant tissues. See, e.g., K. E. Fujimura, et al., Role of the gut microbiota in defining human health, 8 EXPERT REVIEW OF ANTI-INFECTIVE THERAPY 435 (2010); A. S. Neish, Microbes in gastrointestinal health and disease, 136 GASTROENTEROLOGY 65 (2009); J. C. Clemente, et al., The impact of the gut microbiota on human health: an integrative view, 148 CELL 1258 (2012); M. C. Noverr & G. B. Huffnagle, Does the microbiota regulate immune responses outside the gut?, 12 TRENDS IN MICROBIOLOGY 562 (2004); H. Tlaskalova-Hogenova, et al., The role of gut microbiota (commensal bacteria) and the mucosal barrier in the pathogenesis of inflammatory and autoimmune diseases and cancer: contribution of germ free and gnotobiotic animal models of human diseases, 8 CELLULAR & MOLECULAR IMMUNOLOGY 110 (2011); each of which is incorporated by reference herein in its entirety. Along these lines, experimental and clinical studies have shown that the dietary enrichment with certain “probiotic” organisms activates immune and metabolic pathways that restore tissue homeostasis and promote overall health. See, e.g., J. Ravel, et al., Vaginal microbiome of reproductive-age women, 108 (Suppl. 1) PROCEEDINGS NAT'L ACAD. SCI. USA 4680 (2011); A. A. Litonjua & S. T. Weiss, Is vitamin D deficiency to blame for the asthma epidemic?, 120 J. ALLERGY & CLINICAL IMMUNOLOGY 1031 (2007); M. H. Floch, et al., Recommendations for probiotic use-2011 update, 45 (Suppl.) J. CLINICAL GASTROENTEROLOGY S168 (2011); each of which is incorporated by reference herein in its entirety.

The probiotic concept has been well established in human and animal health during the past century. Bacillus spp. have been widely used as probiotic ingredient in animal feed products, in human dietary and over-the-counter medicinal supplements and are even consumed as food ingredients (Hong, et al., “The use of bacterial spore formers as probiotics,” FEMS Microbiology Reviews (2005) 29:813-835). The most extensively studied probiotics belonging to the Bacillus genus include Bacillus subtilis, B. clausii, B. coagulans and B. licheniformis (Cutting, S. M., “Bacillus probiotics,” Food Microbiology (2011) 28:214-220). These species are gram-positive bacteria able to form endospores, which are robust entities that are able to survive extremes of temperature, irradiation and long-term storage (Nicholson, W. L., “Roles of Bacillus endospores in the environment,” Cellular and Molecular Life Sciences: CMLS (2002) 59:410-416). Furthermore, they are adapted to survive in the host gastrointestinal tract as they are resistant to gastric acidity, resulting in the delivery of highly viable probiotics in the small intestine, where germination and further proliferation occurs, a phenomenon proven by several in vivo molecular studies (Tam, N. K. M.; et al., “The Intestinal Life Cycle of Bacillus subtilis and Close Relatives,” Journal of Bacteriology (2006) 188:2692-2700; Cartman, S. T., et al., “Bacillus subtilis Spores Germinate in the Chicken Gastrointestinal Tract,” Applied and Environmental Microbiology (2008) 74:5254-5258; Casula, G. and Cutting, S. M., “Bacillus Probiotics: Spore Germination in the Gastrointestinal Tract,” Applied and Environmental Microbiology (2002) 68:2344-2352).

Numerous studies have explored potential health benefits of Bacillus strains. It was reported that several Bacillus strains exert immunomodulatory properties upon germination in the gut. Germinating spores of Bacillus subtilis var. Natto, for instance, secrete a serine protease (nattokinase) which has been linked with reduction of blood clotting by fibrinolysis. Furthermore, Rhee et al. (Rhee, K. J.; et al., “Role of commensal bacteria in development of gut-associated lymphoid tissues and preimmune antibody repertoire,” Journal of Immunology (Baltimore, Md. 1950) (2004) 172:1118-1124) reported that orally administered Bacillus subtilis spores stimulated the development of gut-associated lymphoid tissue (GALT) in infant rabbits upon germination and re-sporulation. Next to immunomodulatory properties, Bacillus strains are known for the secretion of antimicrobial compounds thereby exerting an anti-pathogenic effect. Bacillus subtilis, for instance, suppressed pathogenic infection with Salmonella enterica, Clostridium perfringens and Escherichia coli in a poultry model (La Ragione, R. M. and Woodward, M. J., “Competitive exclusion by Bacillus subtilis spores of Salmonella enterica serotype Enteritidis and Clostridium perfringens in young chickens,” Veterinary Microbiology (2003) 94:245-256; and La Ragione, et al., “Bacillus subtilis spores competitively exclude Escherichia coli O78:K80 in poultry,” Veterinary Microbiology (2001) 79:133-142). Additionally, suppression of the mouse enteropathogen Citrobacter rodentium was reported following the administration of Bacillus subtilis spores (D'Arienzo, R.; et al., “Bacillus subtilis spores reduce susceptibility to Citrobacter rodentium-mediated enteropathy in a mouse model,” Research in Microbiology (2006) 157:891-897).

Probiotics are most commonly defined as “live microorganisms which when administered in adequate amounts confer a health benefit on the host,” such as restoring or improving the composition of intestinal microflora. See, e.g., FAO/WHO, Guidelines for the evaluation of probiotics in food, London, Ontario, Canada (2002), incorporated by reference herein in its entirety. Probiotics are typically provided as dietary supplements containing potentially beneficial bacteria or yeast and are widely consumed in foods, including dairy products and probiotic fortified foods, as well as in capsules, tablets, and powders. See, e.g., C. Stanton, et al., Market potential of probiotics, 73 (Suppl.) AM. J. CLINICAL NUTRITION 476S (2001), incorporated by reference herein in its entirety. It is believed by many experts that the ideal probiotic should remain viable at the level of the intestine and should adhere to the intestinal epithelium to confer a significant health benefit. There is some evidence to support the importance of viability in human studies, with viable bacteria having greater immunological effects that nonviable bacteria. See, e.g., M. Kaila, et al., Viable versus inactivated lactobacillus strain GG in acute rotavirus diarrhea, 72 ARCHIVES OF DISEASE IN CHILDHOOD 51 (1995); P. V. Kirjavainen, et al., Probiotic bacteria in the management of atopic disease: underscoring the importance of viability, 36 J. PEDIATRIC GASTROENTEROLOGY & NUTRITION 223 (2003); each of which is incorporated by reference herein in its entirety. Some of the best characterized probiotics have also been shown to adhere strongly to intestinal epithelium in both in vitro and in vivo studies. See, e.g., M. Alander, et al., Persistence of colonization of human colonic mucosa by a probiotic strain, Lactobacillus rhamnosus GG, after oral consumption, 65 APPLIED & ENVIRONMENTAL MICROBIOLOGY 351 (1999), incorporated by reference herein in its entirety. Probiotics must also be resistant to gastric acid digestion and to bile salts to reach the intestine intact, and they should be nonpathogenic. Most probiotics are strains of lactic acid bacteria, including Lactobacillus and Bifidobacterium species. Some have been isolated from the intestinal microbiota of healthy humans; others have been isolated from fermented dairy products. Species and strains from other bacterial genera such as Streptococcus, Bacillus, Enterococcus, Lactococcus, Propionibacterium, Saccharomyces, and Escherichia have also been used as probiotics or have been reported to have probiotic properties, but there are concerns surrounding the safety of some of these probiotics because they contain many pathogenic species, particularly within the genus Enterococcus. Nonbacterial microorganisms such as yeasts from the genus Saccharomyces have also been used as probiotics for many years.

Recently, extensive research has been conducted on the potential involvement of the gut microbiome in host metabolism (Cani, P. D. and Delzenne, N. M., “The role of the gut microbiota in energy metabolism and metabolic disease,” Current Pharmaceutical Design (2009) 15:1546-1558). A key area of interest is the role of the gut microbiota in the occurrence of metabolic diseases, such as obesity and obesity-related disorders such as type 2 diabetes. It has been suggested that the gut microbiome contributes to the occurrence of these metabolic diseases by the regulation of endotoxemia, the latter being a state characterized by elevated circulating lipopolysaccharide (LPS) levels (Cani, P. D.; et al., “Selective increases of bifidobacteria in gut microflora improve high-fat-diet-induced diabetes in mice through a mechanism associated with endotoxaemia,” Diabetologia (2007) 50:2374-83). A link between elevated LPS levels in plasma, and the presence of obesity and obesity-related disorders has been established by several researchers over the past years (Cani, P. D.; et al., “Metabolic endotoxemia initiates obesity and insulin resistance,” Diabetes (2007) 56:1761-72; Creely, S. J.; et al., “Lipopolysaccharide activates an innate immune system response in human adipose tissue in obesity and type 2 diabetes,” American Journal of Physiology, Endocrinology and Metabolism (2007) 292: E740-7; and Manco, M.; et al., “Gut microbiota, lipopolysaccharides, and innate immunity in the pathogenesis of obesity and cardiovascular risk,” Endocrine Reviews (2010) 31:817-44). The impact of modulation of the gut microbiota on metabolic endotoxemia has therefore been proposed as a potential strategy in the control of metabolic disease development. González-Sarrías et al. showed a significant association between decreased endotoxemia in obese individuals and increased levels of Faecalibacterium and Odoribacter, and reduction of Parvimonas levels (Gonzalez-Sarrias, A.; et al., “The Endotoxemia Marker Lipopolysaccharide-Binding Protein is Reduced in Overweight-Obese Subjects Consuming Pomegranate Extract by Modulating the Gut Microbiota: A Randomized Clinical Trial,” Molecular nutrition &food research (2018) 62: e1800160). Furthermore, Cani et al. (Diabetologia, 2007) reported a significant correlation between elevated colonic Bifidobacterium levels and decreased endotoxemia in high-fat diet-fed mice upon consumption of the prebiotic compound oligofructose. Recently, it was reported that supplementation of an oral spore-based probiotic supplement, containing a mixture of five Bacillus strains, reduced post-prandial metabolic endotoxemia in human subjects, indicating a potential role of probiotics in targeting metabolic diseases (McFarlin, B. K.; et al., “Oral spore-based probiotic supplementation was associated with reduced incidence of post-prandial dietary endotoxin, triglycerides, and disease risk biomarkers,” World Journal of Gastrointestinal Pathophysiology (2017) 8:117-126).

In vitro approaches are widely used to study possible changes in the intestinal microbial community upon supplementation of test substances. If a way could be found to produce spore-based probiotic compositions, and use said compositions to protect against obesity-related disorders, this would represent a useful contribution to the art.

### SUMMARY OF THE INVENTION

In an embodiment, the present disclosure relates to a method of administration of a spore-based probiotic composition for modulating microbiome and/or microbiota in a human subject.

In another embodiment, a method is described for modulating microbial metabolic activity or microbial community composition in a human subject, including administering to the human subject an effective amount of a spore-based probiotic composition comprising strains Bacillus indicus (HU36), Bacillus subtilis (HU58), Bacillus coagulans SC-208, Bacillus clausii SC-109, and Bacillus licheniformis SL-307, each strain comprising Bacillus spores, wherein a health outcome is improved in the human subject. Health outcomes include, but are not limited to, protection against a condition selected from the group consisting of obesity-related disorders, metabolic disorders, inflammation, and cancer.

In another embodiment, a method is described for increasing microbial diversity in the gastro-intestinal tract in a human subject, including administering to the human subject an effective amount of a spore-based probiotic composition comprising strains Bacillus indicus (HU36), Bacillus subtilis (HU58), Bacillus coagulans SC-208, Bacillus clausii SC-109, and Bacillus licheniformis SL-307, each strain comprising Bacillus spores. Exemplary increases in colonization of Bifidobacteriaceae, Faecalibacterium prausnitzii, and Akkermansia muciniphila were observed, among other beneficial microbial species.

In another embodiment, specific intestinal behavior is found for Bacillus indicus HU-36 with efficient germination and survival, which translates to a positive modulation of the intestinal environment by the strain.

In another embodiment, specific intestinal behavior is found for Bacillus indicus HU-58 with efficient germination and survival, which translates to a positive modulation of the intestinal environment by the strain.

### BRIEF DESCRIPTION OF THE DRAWINGS

FIG. 1 depicts, in one embodiment, microbial metabolic activity in terms of SCFA production in samples of three (3) human donors. Average acetate (A-C; mM), propionate (D-F; mM) and butyrate (G-I; mM) production during the control (C1-C2; n=3) and the treatment (TR1-3; n=3) weeks in the ascending (AC), transverse (TC) and descending colon (DC) of the human gastro-intestinal tract for three donors tested. Data is presented as mean±stdev. Statistically significant differences relative to the first control week are indicated with * (p<0.05).

FIG. 2 depicts, in one embodiment, microbial community composition in samples of the three (3) human donors as assessed via 16S-targeted Illumina Sequencing. Abundance (%) at microbial phylum level in the ascending colon (AC) of the human gastro-intestinal tract for three donors tested over the control (C) and the treatment (TR) period. For optimal observation of consistent effects, the average of the control period (n=2) and the average of the final two weeks of treatment (n=2) are presented.

FIG. 3 depicts, in one embodiment, Average Reciprocal Simpson Diversity Index in the ascending (AC), transverse (TC) and descending colon (DC) reactors during the control (C) and two final treatment (TR) weeks upon supplementation of the probiotic formulation for three different human donors tested (n=2). Statistical differences between control and treatment, as calculated with a 2-sided Student T-test are indicated by the respective p-values. P-values<0.05 are indicated with * (p<0.05).

FIG. 4 depicts the measured effects of MegaSporeBiotic on the luminal Firmicutes:Bacteroidetes ratio in the ascending (AC), transverse (TC) and descending colon (DC) reactors. Left panel: average levels during the control (C) and treatment (TR) weeks (bars for each colon segment read left to right as C1, C2, TR1, TR2, TR3). Right panel: average levels over the entire control and treatment period (bars for each colon segment read left to right as C, TR). * indicates statistically significant differences relative to the preceding period.

FIG. 5 depicts Average Reciprocal Simpson Diversity Index together with abundances (%) of bacterial phyla in the ascending (AC), transverse (TC) and descending colon (DC) reactors (n=5). For optimal visualization, within each parameter, the intensity of background shading for a given colon compartment is correlated to the absolute value of that parameter. Statistical differences between colon regions, as calculated with a 2-sided student t-test, are indicated by the respective p-values. P-values≤0.05 are indicated in bold.

FIG. 6 depicts Average abundances (%) of bacterial families in the ascending (AC), transverse (TC) and descending colon (DC) reactors (n=5). For optimal visualization, within each family, the intensity of background shading for a given colon compartment is correlated to the absolute abundance of that family. Statistical differences between colon regions, as calculated with a 2-sided student t-test, are indicated by the respective p-values. P-values≤0.05 are indicated in bold.

FIG. 7 depicts Average Reciprocal Simpson Diversity Index together with abundances (%) of bacterial phyla in the ascending (AC), transverse (TC) and descending colon (DC) reactors during the control period (C) and two final treatment (TR) weeks with MegaSporeBiotic (n=2). For optimal visualization, within each parameter, the intensity of background shading for a given colon compartment and treatment group (C or TR) is correlated to the absolute value of that parameter. Statistical differences between control and treatment (C vs TR), as calculated with a 2-sided student t-test, are indicated by the respective p-values. P-values≤0.05 are indicated in bold.

FIG. 8 depicts Average abundances (%) of bacterial families in the ascending (AC), transverse (TC) and descending colon (DC) reactors during the control period (C) and two final treatment (TR) weeks with MegaSporeBiotic (n=2). For optimal visualization, within each family, the intensity of background shading for a given colon compartment and treatment group (C or TR) is correlated to the absolute value of that family. Statistical differences between control and treatment (C vs TR), as calculated with a 2-sided student t-test, are indicated by the respective p-values. P-values≤0.05 are indicated in bold.

FIG. 9 depicts Average abundances of a selection of OTUs (related to a certain species), based on reported changes at family/phylum level in the ascending (AC), transverse (TC) and descending colon (DC) reactors during the control period (C) and two final treatment (TR) weeks with MegaSporeBiotic (n=2). For optimal visualization, within each OTU, the intensity of background shading for a given colon compartment and treatment group (C or TR) is correlated to the absolute value of that OTU. Statistical differences between control and treatment (C vs TR), as calculated with a 2-sided student t-test, are indicated by the respective p-values. P-values≤0.05 are indicated in bold.

FIG. 10 depicts effects of the SHIME-collected samples on (A) IL-1β, (B) IL-8, (C) CXCL10, (D) TNF-α and (E) MCP-1 levels. Cytokine levels were measured after 6 h of LPS treatment of the co-cultures that were first pre-treated for 24 h with SHIME-collected samples. The red dotted line corresponds to the experimental control LPS+. (*) represents statistical significant differences between control and treatment with MegaSporeBiotic.

FIG. 11 depicts effects of the SHIME-treatment samples on (A) TEER, (B) IL-1b, TNF-α and NFkb, (C) MCP-1, IL-8 and CXCL10, (D) (E) IL-10 and IL-6 levels, after normalization to the respective controls. Cytokine levels were measured after 6 h of LPS treatment of the co-cultures that were first pre-treated for 24 h with SHIME-collected samples. Concentrations of the treatment samples with MegaSporeBiotic were normalized to the respective SHIME control. The dotted line corresponds to 100%.

### DETAILED DESCRIPTION

A spore-based probiotic composition is described that includes at least one viable probiotic microorganism having a biological or therapeutic on microbiome in humans. One exemplary composition contains five different strains of Bacillus spp. Also provided are methods of producing spore-based probiotic compositions. A validated in vitro gut model adapted from Molly, et al., “Development of a 5-step multi-chamber reactor as a simulation of the human intestinal microbial ecosystem,” Applied Microbiology and Biotechnology (1993) 39:254-258 (i.e. SHIME®), herein incorporated by reference in its entirety, was used to assess the long-term effect of the composition on microbial metabolic activity and community composition. The results support use of the composition in protecting against obesity-related disorders.

In the present study, the gut-modulatory effect of a probiotic composition containing a mixture of five probiotic Bacillus strains in a standardized in vitro simulation of the intestinal environment was evaluated. This experimental design allowed insight into the impact of the probiotic formulation on microbial activity and composition changes of three human individuals, with the aim of highlighting the potential mechanism-of-action behind the effects observed in vivo (See, McFarlin, et al., 2017).

As used herein, the verb “comprise” as is used in this description and in the claims and its conjugations are used in its non-limiting sense to mean that items following the word are included, but items not specifically mentioned are not excluded. In addition, reference to an element by the indefinite article “a” or “an” does not exclude the possible that more than one of the elements are present, unless the context clearly requires that there is one and only one of the elements. The indefinite article “a” or “an” thus usually means “at least one.”

As used herein, an “effective amount” or an “amount effective for” is defined as an amount effective, at dosages and for periods of time necessary, to achieve a desired biological result, such as reducing, preventing, or treating a disease or condition and/or inducing a particular beneficial effect. The effective amount of compositions of the disclosure may vary according to factors such as age, sex, and weight of the individual. Dosage regime may be adjusted to provide the optimum response. Several divided doses may be administered daily, or the dose may be proportionally reduced as indicated by the exigencies of an individual's situation. As will be readily appreciated, a composition in accordance with the present disclosure may be administered in a single serving or in multiple servings spaced throughout the day. As will be understood by those skilled in the art, servings need not be limited to daily administration, and may be on an every second or third day or other convenient effective basis. The administration on a given day may be in a single serving or in multiple servings spaced throughout the day depending on the exigencies of the situation.

As used herein, the term “subject” or “individual” refers to any vertebrate including, without limitation, humans and other primates (e.g., chimpanzees and other apes and monkey species), farm animals (e.g., cattle, sheep, pigs, goats, and horses), domestic animals (e.g., dogs and cats), laboratory animals (e.g., rodents such as mice, rats, and guinea pigs), and birds (e.g., domestic, wild, and game birds such as chickens, turkeys, and other gallinaceous birds, ducks, geese, and the like). In some implementations, the subject may be a mammal. In other implementations, the subject may be a human.

A validated in vitro gut model, which is a simulated human intestinal microbial ecosystem (i.e., SHIME®), was used to assess the long-term effect of a spore-based probiotic formulation, containing five different Bacillus strains, on microbial metabolic activity and community composition, taking into account the issue of inter-individual variability. First, a colon-region specific colonization was demonstrated with increasing microbial diversity from the ascending to the distal colon regions. Furthermore, a donor-dependent modulation of microbial metabolism and composition was revealed, with the main effects being observed in the distal colon. The test product significantly increased levels of acetate and propionate, with strongest effects observed for donor 2 (on average +13.0 mM acetate and +5.6 mM propionate in the distal colon areas), whereas donor 3 was mainly characterized by increased propionate levels (+1.0 mM). Donor 1 showed a different metabolic profile, as repeated intake of the probiotic formulation resulted in increased butyrate over propionate levels. Bifidobacteriaceae were found to increase for all three donors tested. Particularly two organizational taxonomic units (“OTUs”) related to Bifidobacterium adolescentis and Bifidobacterium bifidum increased upon supplementation of the probiotic formulation. Moreover, for donor 1 and 2 Faecalibacterium prausnitzii increased, whereas for donor 3 the health-promoting Akkermansia muciniphila was enriched upon probiotic supplementation. The generated data support a possible role of the test product in protecting against obesity-related disorders.

In embodiments of the present invention, the probiotic compositions may contain a probiotic microorganism that in some applications may be a spore-based probiotic organism selected from the following genera: Lactobacillus, Bifidobacterium (i.e., of Family Bifidobacteriaceae), Lactococcus, Propionibacterium, Bacillus, Akkermansia, Faecalibacterium, Enterococcus, Escherichia, Streptococcus, Pediococcus, and Saccharomyce. In certain aspects, the probiotic microorganism is at least one of Lactobacillus acidophilus, Lactobacillus rhamnosus, Lactobacillus fermentum, Lactobacillus casei, Lactobacillus bulgaricus, Lactobacillus gasseri, Lactobacillus helveticus, Lactobacillus johnsonii, Lactobacillus lactis, Lactobacillus plantarum, Lactobacillus reuteri, Lactobacillus salivarius, Lactobacillus paracasei, Bifidobacterium sp., Bifidobacterium longum, Bifidobacterium infantis, Bifidobacterium animalis, Bifidobacterium bifidum, Bifidobacterium adolescentis, Bifidobacterium lactis, Bacillus subtilis, Bacillus coagulans, Bacillus licheniformis, Akkermansia muciniphila, Faecalibacterium prausnitzii, Enterococcus faecalis, Enterococcus faecium, Lactococcus lactis, Streptococcus salivarius, Saccharomyces cerevisiae, and Saccharomyces boulardii. The probiotic microorganism may be in the form of spores or in a vegetative state.

In one embodiment, the spore-containing compositions may or may not contain one or more of the above bacterial species, and yet said compositions may be used to increase the growth of those protective, beneficial bacterial populations by adding the spore-containing composition, thus increasing the overall microbiome diversity.

The Lactobacillus genus is extremely diverse and expanding every year. With over 230 species, it has grown into one of the biggest genera in the bacterial taxonomy. As the genus has exceeded the acceptable “normal diversity,” renaming and re-classification is inevitable wherein the genus Lactobacillus may be split into most likely twelve new genera. Many traditional “probiotic” species with substantiated industrial importance and starter cultures many no longer eventually be called “Lactobacillus.” Hence, a substantial communication challenge looms ahead to reduce the inevitable confusion regarding the “old commercial” and “correct scientific” nomenclature. Once the International Committee on Systematics of Prokaryotes publishes new nomenclature in their official journal, the INTERNATIONAL JOURNAL OF SYSTEMATIC AND EVOLUTIONARY MICROBIOLOGY, the changes are valid and official. The manuscript that will be submitted for publication outlining the new nomenclature of the Lactobacillus genus will likely be ready for submission by the end of 2018. Meanwhile, there was a taxonomic subcommittee meeting in September 2018 to discuss the nomenclature changes and an (invite-only) expert LABIP workshop in October 2018 that will evaluate the science while considering the consequences for regulations, legal/IP, and industry.

Probiotics are measured by colony forming units (“CFUs”) and can be measured as CFUs/g or CFUs/vol. Alternatively, a given probiotic dosage can be delivered as a total in CFUs. Few studies have been done to determine effective dosages, but effective dosages are usually in the hundreds of millions of CFUs or higher. If probiotics are being used to help with digestion, probiotics should be taken with meals, but otherwise the probiotics may survive better if taken between meals, particularly if taken with liquids that help to dilute stomach acid and move the probiotics more quickly into the digestive tract. Probiotics may be given short-term or long-term.

In some implementations, the concentration of the probiotic microorganism in the composition may be at least about 1·10⁹ CFU/g, at least about 2·10⁹ CFU/g, at least about 3·10⁹ CFU/g, at least about 4·10⁹ CFU/g, at least about 5·10⁹ CFU/g, at least about 6·10⁹ CFU/g, at least about 7·10⁹ CFU/g, at least about 8·10⁹ CFU/g, at least about 9·10⁹ CFU/g, at least about 1·10¹⁰ CFU/g, at least about 2·10¹⁰ CFU/g, at least about 3·10¹⁰ CFU/g, at least about 4·10¹⁰ CFU/g, at least about 5·10¹⁰ CFU/g, at least about 6·10¹⁰ CFU/g, at least about 7·10¹⁰ CFU/g, at least about 8·10¹⁰ CFU/g, at least about 9·10¹⁰ CFU/g, or at least about 1·10¹¹ CFU/g.

The spore-based probiotic supplement may comprise spores having a survival rate within any of the following ranges after exposure to gastric acid in situ: about 75% to about 99%, about 80% to about 95%, about 85% to about 90%, and greater than about 90%.

The spore-based probiotic supplement may comprise a number of spores within any of the following ranges: about 1 billion to about 10 billion spores, about 1.5 billion spores to about 9.5 billion spores, about 2 billion spores to about 9 billion spores, about 2.5 billion spores to about 8 billion spores, about 3 billion spores to about 7 billion spores, about 3.5 billion spores to about 6.5 billion spores, about 3.5 billion spores to about 6 billion spores, about 3.5 billion spores to about 5 billion spores, and about 3.5 billion spores to about 4.5 billion spores.

The spore-based probiotic supplement may comprise a liquid, confectionary item, powder or pill form or may be added to a food product. In one implementation, about 1·10¹⁰ CFU of microorganism is present in each gram of bulk, dried raw powder where each gram contains about 60% or less of bacterial mass and about 40% carrier system. In other implementations, each gram contains about 70% or less of bacterial mass and about 30% carrier system, about 80% or less of bacterial mass and about 20% carrier system, about 90% or less of bacterial mass and about 10% carrier system, about 50% or less of bacterial mass and about 50% carrier system, about 40% or less of bacterial mass and about 60% carrier system, about 30% or less of bacterial mass and about 70% carrier system, about 20% or less of bacterial mass and about 80% carrier system, or about 10% or less of bacterial mass and about 90% carrier system.

Implementations of the methods and compositions disclosed herein may comprise a spore-based probiotic. A spore-based probiotic is comprised of endosomes which are highly resistant to acidic pH, are stable at room temperature, and deliver a much greater quantity of high viability bacteria to the small intestine than traditional probiotic supplements. Traditional micro-encapsulation uses live microorganisms which are then micro-encapsulated in an effort to protect the microorganisms; however, this is a process that inherently leads to the eventual death of the microorganisms thereby reducing the efficacy of the microorganisms. Using spore-based microorganisms that have been naturally microencapsulated to form endosomes may be preferable as these microorganisms are dormant and do not experience a degradation in efficacy over time. These spore-based microorganisms are also particularly thermally stable and can survive UV pasteurization, so they are also able to be added to food products or beverages prior to thermal exposure or UV pasteurization without experiencing a degradation in efficacy over time.

Micro-Encapsulation

In certain implementations, the probiotic microorganisms are microencapsulated prior to addition to the probiotic compositions. Micro-encapsulation is a process in which tiny particles or droplets are surrounded by a coating to give small capsules of many useful properties. In a relatively simple form, a microcapsule is a small sphere with a uniform wall around it. The material inside the microcapsule is referred to as the core, internal phase, or fill, whereas the wall is sometimes called a shell, coating, or membrane. Most microcapsules have diameters between a few micrometers and a few millimeters.

The definition of “microencapsulation” has been expanded, and includes most foods. Every class of food ingredient has been encapsulated; flavors are the most common. The technique of microencapsulation depends on the physical and chemical properties of the material to be encapsulated. See, e.g., L. S. Jackson & K. Lee, Microencapsulation and the food industry, LEBENSMITTEL-WISSENSCHAFT TECHNOLOGIE (Jan. 1, 1991), incorporated by reference herein in its entirety.

Many microcapsules, however, bear little resemblance to these simple spheres. The core may be a crystal, a jagged absorbent particle, an emulsion, a Pickering emulsion, a suspension of solids, or a suspension of smaller microcapsules. The microcapsule even may have multiple walls.

Various techniques may be used to produce microcapsules, and each of such various techniques will be understood by a person of ordinary skill in the art. These techniques that may be used to produce microcapsules include, but are not limited to, pan coating, air-suspension coating, centrifugal extrusion, vibrational nozzle, spray-drying, ionotropic gelation, interfacial polycondensation, interfacial cross-linking, in situ polymerization, and matrix polymerization, as described below.

Pan Coating

The pan coating process, widely used in the pharmaceutical industry, is among the oldest industrial procedures for forming small, coated particles or tablets. The particles are tumbled in a pan or other device while the coating material is applied slowly.

Air-Suspension Coating

Air-suspension coating, first described by Professor Dale Eavin Wurster at the University of Wisconsin in 1959, gives improved control and flexibility compared to pan coating. In this process, the particulate core material, which is solid, is dispersed into the supporting air stream and these suspended particles are coated with polymers in a volatile solvent leaving a very thin layer of polymer on them. This process is repeated several hundred times until the required parameters such as coating thickness, etc., are achieved. The air stream which supports the particles also helps to dry them, and the rate of drying is directly proportional to the temperature of the air stream which can be modified to further affect the properties of the coating.

The re-circulation of the particles in the coating zone portion is effected by the design of the chamber and its operating parameters. The coating chamber is arranged such that the particles pass upwards through the coating zone, then disperse into slower moving air and sink back to the base of the coating chamber, making repeated passes through the coating zone until the desired thickness of coating is achieved.

Centrifugal Extrusion

Liquids are encapsulated using a rotating extrusion head containing concentric nozzles. In this process, a jet of core liquid is surrounded by a sheath of wall solution or melt. As the jet moves through the air it breaks, owing to Rayleigh instability, into droplets of core, each coated with the wall solution. While the droplets are in flight, a molten wall may be hardened or a solvent may be evaporated from the wall solution. Because most of the droplets are within +10% of the mean diameter, they land in a narrow ring around the spray nozzle. Hence, if needed, the capsules can be hardened after formation by catching them in a ring-shaped hardening bath. This process is excellent for forming particles 400-2,000 μm in diameter. Because the drops are formed by the breakup of a liquid jet, the process is only suitable for liquid or slurry. A high production rate can be achieved, i.e., up to 22.5 kg (50 lb) of microcapsules can be produced per nozzle per hour per head. Heads containing 16 nozzles are available.

Vibrational Nozzle

Core-Shell encapsulation or Microgranulation (matrix-encapsulation) can be done using a laminar flow through a nozzle and an additional vibration of the nozzle or the liquid. The vibration has to be done in resonance of the Rayleigh instability and leads to very uniform droplets. The liquid can consist of any liquids with limited viscosities (0-10,000 mPa·s have been shown to work), e.g., solutions, emulsions, suspensions, melts, etc. The solidification can be done according to the used gelation system with an internal gelation (e.g., sol-gel processing, melt) or an external (additional binder system, e.g., in a slurry). The process works very well for generating droplets between 20-10,000 μm, applications for smaller and larger droplets are known. The units are deployed in industries and research mostly with capacities of 1-20,000 kg per hour (2-44,000 lb/h) at working temperatures of 20-1500° C. (68-2732° F.) (room temperature up to molten silicon). Nozzle heads with from one up to several hundred thousand nozzles are available.

Spray-Drying

Spray drying serves as a microencapsulation technique when an active material is dissolved or suspended in a melt or polymer solution and becomes trapped in the dried particle. The main advantages are the abilities to handle labile materials because of the short contact time in the dryer; in addition, the operation is economical. In modern spray dryers the viscosity of the solutions to be sprayed can be as high as 300 mPa·s. By combining this technique with the use of supercritical Carbon Dioxide, sensitive materials like proteins can be encapsulated.

Ionotropic Gelation

The coacervation-phase separation process consists of three steps carried out under continuous agitation, as follows:

(1) Formation of 3 immiscible chemical phases: liquid manufacturing vehicle phase, core material phase, and coating material phase;

(2) Deposition of coating: core material is dispersed in the coating polymer solution. Coating polymer material coated around core. Deposition of liquid polymer coating around core by polymer adsorbed at the interface formed between core material and vehicle phase;

(3) Rigidization of coating: coating material is immiscible in vehicle phase and it gets rigid in form. Techniques for rigidization include thermal, cross-linking, or dissolvation.

Interfacial Polycondensation

In interfacial polycondensation, the two reactants in a polycondensation meet at an interface and react rapidly. The basis of this method is the classical Schotten-Baumann reaction between an acid chloride and a compound containing an active hydrogen atom, such as an amine or alcohol, a polyester, a polyuria, or a polyurethane. Under the right conditions, thin flexible walls form rapidly at the interface. A solution of the pesticide and a diacid chloride are emulsified in water and an aqueous solution containing an amine and a polyfunctional isocyanate is added. Base is present to neutralize the acid formed during the reaction. Condensed polymer walls form instantaneously at the interface of the emulsion droplets.

Interfacial Cross-Linking

Interfacial cross-linking is derived from interfacial polycondensation, and was developed to avoid the use of toxic diamines, for pharmaceutical or cosmetic applications. In this method, the small bifunctional monomer containing active hydrogen atoms is replaced by a biosourced polymer, like a protein. When the reaction is performed at the interface of an emulsion, the acid chloride reacts with the various functional groups of the protein, leading to the formation of a membrane. The method is very versatile, and the properties of the microcapsules (size, porosity, degradability, mechanical resistance) may be varied. Flow of artificial microcapsules in microfluoridic channels is contemplated.

In-Situ Polymerization

In a few microencapsulation processes, the direct polymerization of a single monomer is carried out on the particle surface. In one process, e.g., cellulose fibers are encapsulated in polyethylene while immersed in dry toluene. Usual deposition rates are about 0.5 μm/min. Coating thickness ranges 0.2-75 μm (0.0079-3.0 mils). The coating is uniform, even over sharp projections. Protein microcapsules are biocompatible and biodegradable, and the presence of the protein backbone renders the membrane more resistant and elastic than those obtained by interfacial polycondensation.

Matrix Polymerization

In a number of processes, a core material is imbedded in a polymeric matrix during formation of the particles. A simple method of this type is spray-drying, in which the particle is formed by evaporation of the solvent from the matrix material. However, the solidification of the matrix also can be caused by a chemical change.

This invention is further illustrated by the following additional examples that should be construed as limiting. Those of skill in the art should, in light of the present disclosure, appreciate that many changes can be made to the specific embodiments that are disclosed and still obtain a like or similar result without departing from the spirit and scope of the invention.

### EXAMPLES

All chemicals were obtained from Sigma-Aldrich (Overijse, Belgium) unless stated otherwise. Microbiome Labs (Glenview, USA) provided the probiotic formulation (MegaSporeBiotic), a probiotic formula (containing 5 strains) containing 4×10⁹ spores from the Bacillus indicus (HU36), Bacillus subtilis (HU58), Bacillus coagulans SC-208, Bacillus licheniformis SL-307, and Bacillus clausii SC-109, per capsule. The probiotic formulation was tested at a dose of 2 capsules per day.

HU36 (“Colorspore™”) is a strain of Bacillus indicus, a preparation of which is manufactured by Viridis BioPharma Pvt. Ltd., Mumbai, India. The National Collection of Industrial, Food and Marine Bacteria (“NCIMB”) Ltd. assigned strain number for Bacillus indicus HU36 is 41361.

HU58 (“ProBiotene™”) is a strain of Bacillus subtilis, a preparation of which is manufactured by Viridis BioPharma Pvt. Ltd., Mumbai, India. Bacillus subtilis HU58 has been deposited with the National Center for Biotechnology Research under the accession number EF101709. The Bacillus Genetic Stock Center (“BGSC”) assigned number for Bacillus HU58 is 3A34, and the NCIMB Ltd. assigned strain number is 30283.

SC-109 is a strain of Bacillus clausii, a preparation of which was manufactured by Synergia Life Sciences Pvt. Ltd., Mumbai, India in March 2018. Bacillus clausii SC-109 has been deposited with the Liebniz Institute DSMZ-German Collection of Microorganisms and Cell Cultures under the accession number DSM 32639.

SC-208 is a strain of Bacillus coagulans, a preparation of which was manufactured by Synergia Life Sciences Pvt. Ltd., Mumbai, India in March 2018. Bacillus coagulans SC-208 has been deposited with the Liebniz Institute DSMZ-German Collection of Microorganisms and Cell Cultures under the accession number DSM 32640.

SL-307 is a strain of Bacillus licheniformis used in the probiotic formulation, a preparation of which was manufactured by Synergia Life Sciences Pvt. Ltd., Mumbai, India.

Simulated Human Intestinal Microbial Ecosystem (SHIME®)

The reactor setup was adapted from the SHIME® reactor (ProDigest and Ghent University, Belgium), as was described by Molly et al. (1993), and represents the gastrointestinal tract of a healthy adult human. It consists of a succession of five reactors simulating the different parts of the human gastrointestinal tract, i.e. the stomach, small intestine and three colon regions, respectively. Upon inoculation with fecal microbiota from a healthy human volunteer, the colon compartments simulate the ascending (AC), transverse (TC) and descending (DC) colon. During the present study, three SHIME experiments were conducted using the fecal microbiota of three different human individuals (male, 34y; female, 28y and male, 33y). Inoculum preparation, retention times, pH, temperature settings and reactor feed composition were previously described in Possemiers, S., et al., “PCR-DGGE-based quantification of stability of the microbial community in a simulator of the human intestinal microbial ecosystem,” FEMS Microbiology Ecology (2004) 49: 495-507. The experimental setup of the SHIME run included a two-week stabilization period, a two-week reference period and a three-week treatment period, as previously described (Van de Wiele, T., et al., “Prebiotic effects of chicory inulin in the simulator of the human intestinal microbial ecosystem,” FEMS Microbiology Ecology (2004) 51:143-153). During the treatment period, the test product was administered daily with the feed at a dose of 2 capsules per day.

Microbial Metabolic Activity

During the reference and treatment period of the SHIME experiment, samples for microbial metabolic activity were collected three times per week from each colon vessel. Short chain fatty acid (SCFA) levels, including acetate, propionate, butyrate and branched SCFA (isobutyrate, isovalerate and isocaproate), were monitored as described in De Weirdt, R, et al., “Human faecal microbiota display variable patterns of glycerol metabolism,” FEMS Microbiology Ecology (2010) 74: 601-611. Lactate quantification was performed using a commercially available enzymatic assay kit (R-Biopharm, Darmstadt, Germany) according to manufacturer's instructions. The effect of the test product on colonic acidification was indirectly measured by calculating the difference in the amount of base (NaOH) and acid (HCl) consumed to maintain the pH of each colon reactor in the correct range.

Microbial Community Analysis

During the reference and treatment period, samples for microbial community analysis were collected once per week from each colon vessel. DNA was isolated using the protocol as described in Vilchez-Vargas, R., et al., “Analysis of the microbial gene landscape and transcriptome for aromatic pollutants and alkane degradation using a novel internally calibrated microarray system,” Environmental Microbiology (2013) 15: 1016-39, starting from pelleted cells originating from 1 mL luminal sample.

Subsequently, quantitative polymerase chain reaction (qPCR) for Akkermansia muciniphila and Faecalibacterium prausnitzii were performed on a QuantStudio 5 Real-Time PCR system (Applied Biosystems, Foster City, Calif., USA). Each sample was analyzed in technical triplicate and outliers (more than 1 CT difference) were omitted. The qPCR for Akkermansia muciniphila was previously described by Collado, M. C., et al., “Intestinal Integrity and Akkermansia muciniphila, a Mucin-Degrading Member of the Intestinal Microbiota Present in Infants, Adults, and the Elderly,” Applied and Environmental Microbiology (2007) 73:7767-7770, while the qPCR for Faecalibacterium prausnitzii was conducted as described in Sokol, H, et al., “Low counts of Faecalibacterium prausnitzii in colitis microbiota,” Inflammatory Bowel Diseases (2009) 15:1183-9.

The microbiota profiling of each colon compartment was established by 16S-targeted sequencing analysis. The 16S rRNA gene V3-V4 hypervariable regions were amplified by PCR using primers 341F (5′-CCT ACG GGN GGC WGC AG-3′) and 785Rmod (5′-GAC TAC HVG GGT ATC TAA KCC-3′), with the reverse primer being adapted from Klindworth, A., et al., “Evaluation of general 16S ribosomal RNA gene PCR primers for classical and next-generation sequencing-based diversity studies,” Nucleic Acids Research (2013) 41:e1-e1, to increase coverage. Quality control PCR was conducted using Taq DNA Polymerase with the Fermentas PCR Kit according to the manufacturers' instructions (Thermo Fisher Scientific, Waltham, Mass., USA). The obtained PCR product was run along the DNA extract on a 2% agarose gel for 30 minutes at 100V. 10 μl of the original genomic DNA extract was send out to LGC genomics GmbH (Germany) for library preparation and sequencing on an Illumina Miseq platform with v3 chemistry with the primers mentioned above.

Statistics

Comparison of normally distributed data of the different reference and treatment weeks on microbial metabolic markers and microbial community parameters was performed with a Student's T-test for pairwise comparisons. Differences were considered significant if p<0.05.

For the 16S-targeted sequencing analysis, read assembly and cleanup was largely derived from the MiSeq protocol described by the Schloss lab (Schloss, P. D., and Westcott, S. L., “Assessing and improving methods used in operational taxonomic unit-based approaches for 16S rRNA gene sequence analysis,” Appl. Environ. Microbiol. (2011) 77: 3219-26; Kozich, J. J., et al., “Development of a dual-index sequencing strategy and curation pipeline for analyzing amplicon sequence data on the MiSeq Illumina sequencing platform,” Appl. Environ. Microbiol. (2013) 79:5112-20). In brief, mothur (v. 1.39.5) was used to assemble reads into contigs, perform alignment-based quality filtering (alignment to the mothur-reconstructed SILVA SEED alignment, v. 123), remove chimeras, assign taxonomy using a naïve Bayesian classifier (Wang, Q., et al., “Naïve Bayesian Classifier for Rapid Assignment of rRNA Sequences into the New Bacterial Taxonomy,” Applied and Environmental Microbiology (2007) 73: 5261-5267) and RDP release 14 (Cole, J. R., et al., “The Ribosomal Database Project: improved alignments and new tools for rRNA analysis,” Nucleic Acids Research (2009) 37: D141-D145) and cluster contigs into OTUs at 97% sequence similarity. All sequences that were classified as Eukaryota, Archaea, Chloroplasts and Mitochondria were removed. Also, if sequences could not be classified at all they were removed. For each OTU, representative sequences were selected as the most abundant sequence within that OTU. Furthermore, the reciprocal Simpson diversity index was calculated as reviewed by Bent, S. J. and Forney, L. J., “The tragedy of the uncommon: understanding limitations in the analysis of microbial diversity,” The ISME Journal (2008) 2: 689.

Analysis of the Microbial Metabolic Activity

The consumption of acid and base reflects the overall microbial activity throughout the SHIME experiment. Results revealed a donor-dependent microbial profile of the test product (Table 1). Base consumption remained unaffected upon dosing the probiotic formulation to donor 1 in all colon regions. Unlike for donor 1, the supplementation of the test product significantly increased base consumption in the distal colon areas during the final weeks of treatment for both donor 2 and 3. The strongest increase was observed for donor 2, with an average increase of 6.5 mL/day in the TC and 2.2 mL/day in the DC as compared to the control period.

Table 1 shows Overall Metabolic Activity In Terms Of Acid/Base Consumption. Average base-acid consumption (mL/day) over the control (C; n=6) and the treatment (TR; n=9) period in the ascending (AC); transverse (TC) and descending colon (DC) of the human gastro-intestinal tract for three donors upon treatment with the probiotic formulation. Data is presented as mean±stdev. Statistically significant differences relative to the control period are indicated with an asterisk (*) (p<0.05).

TABLE 1

| | Donor 1 | | Donor 2 | | Donor 3 | |
|---|---|---|---|---|---|---|
| | C | TR | C | TR | C | TR |
| AC | 4.82 | 3.94 | 4.20 | 3.07 | 1.43 | 3.18 |
| | 2.02 | 1.98 | 2.61 | 0.88 | 0.37 | 5.89 |
| TC | 26.6 | 27.0 | 16.5 | 23.0 (*) | 2.9 | 6.0 |
| | 3.5 | 3.0 | 2.0 | 5.4 | 1.4 | 5.4 |
| DC | 5.07 | 5.79 | 6.17 | 8.39 (*) | 1.92 | 5.13 |
| | 1.43 | 0.82 | 2.10 | 1.61 | 1.67 | 3.04 |

(*): Statistically significant relative to control period.

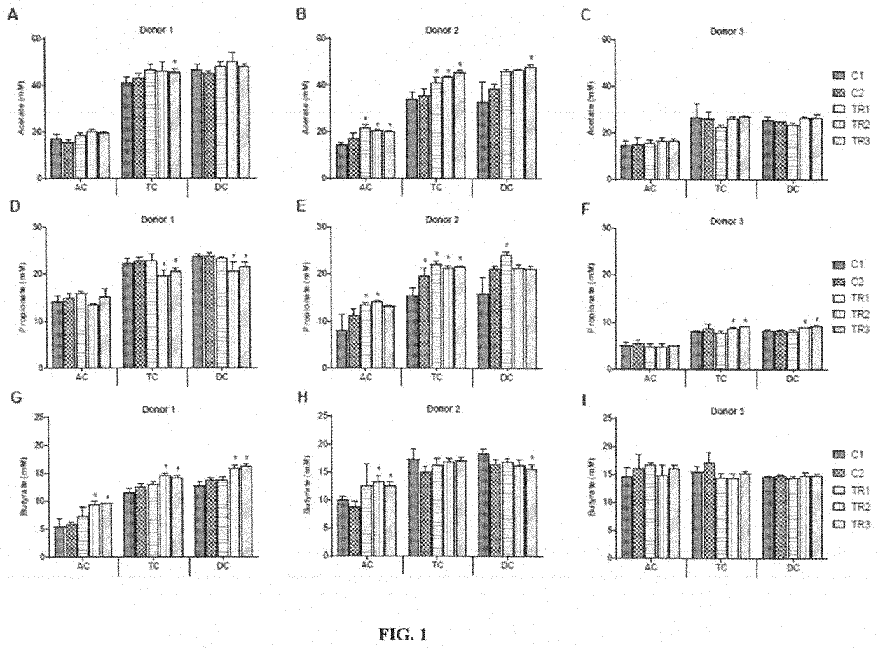

Inter-individual differences were also reflected in the SCFA profiles, which mainly consisted of acetate, propionate and butyrate (FIG. 1) and small amounts of branched SCFA (Table 2). More specifically, supplementation of the probiotic formulation to the microbial community of donor 1 and 2 resulted in statistically significant increases in acetate production compared to the control period in all colon regions, whereas acetate levels remained unaffected for donor 3. The strongest effect was observed for donor 2, with an averagely increased production of acetate of 5.3 mM (+36%), 11.3 mM (+33%) and 14.6 mM (+44%) in the AC, TC and DC respectively. Similar observations were made for butyrate production, with significant increases being observed for donor 1 and 2 during the final weeks of treatment. However, for donor 1 significantly increased butyrate levels were observed in all colon regions, i.e. an average increase of 3.9 mM (+70%) in AC, 2.8 mM (+24%) in TC and 3.6 mM (+28%) in DC as compared to the control period, whereas for donor 2 the butyrogenic effect was only observed in the AC (+2.4 mM (+23%)). As for propionate production, supplementation of the probiotic formulation resulted in significantly decreased propionate production in the distal colon areas (TC and DC) of donor 1 during the final weeks of treatment. For donor 2 on the other hand, enhanced propionate levels were observed in all colon regions, with an average increase of 5.3 mM (+66%), 6.1 mM (+39%) and 5.1 mM (+32%) as compared to the control period in the AC, TC and DC respectively. For donor 3, increased propionate production was only observed in the TC (p<0.001) and DC (p=0.015) during the last two weeks of treatment. Overall, branched SCFA levels were low (Table 3), with a significantly increased production observed in the AC upon dosing the test product to the microbial community of donor 1 and 2, whereas branched SCFA levels remained unaffected for donor 3.

Table 2 shows microbial metabolic activity in terms of LA and branched SCFA production. Average lactate (LA; mM) and branched SCFA (mM) production over the control (C; n=6) and the treatment (TR; n=9) period in the ascending (AC); transverse (TC) and descending colon (DC) of the human gastro-intestinal tract for three donors upon treatment with the probiotic formulation. Data is presented as mean±stdev. Statistically significant differences relative to the control period are indicated with an asterisk (*) (p<0.05).

TABLE 2

| | | Donor 1 | | Donor 2 | | Donor 3 | |
|---|---|---|---|---|---|---|---|
| | | C | TR | C | TR | C | TR |
| Branched | AC | 2.21 | 2.36 (*) | 1.65 | 2.11 (*) | 1.89 | 1.80 |
| SCFA | | 0.07 | 0.08 | 0.20 | 0.11 | 0.26 | 0.11 |
| (mM) | TC | 2.73 | 2.78 | 2.63 | 2.70 | 2.22 | 2.13 |
| | | 0.11 | 0.04 | 0.07 | 0.13 | 0.28 | 0.08 |
| | DC | 2.97 | 2.99 | 2.77 | 2.77 | 2.29 | 2.31 |
| | | 0.07 | 0.08 | 0.09 | 0.07 | 0.04 | 0.08 |
| LA | AC | 0.06 | 0.12 (*) | 0.30 | 0.23 | 0.27 | 0.23 |
| (mM) | | 0.03 | 0.06 | 0.11 | 0.10 | 0.08 | 0.12 |
| | TC | 0.28 | 0.57 (*) | 0.44 | 0.44 | 0.11 | 0.06 (*) |
| | | 0.06 | 0.15 | 0.13 | 0.12 | 0.05 | 0.03 |
| | DC | 0.55 | 0.74 (*) | 0.70 | 0.70 | 0.19 | 0.14 |
| | | 0.06 | 0.12 | 0.17 | 0.13 | 0.07 | 0.08 |

(*): Statistically significant relative to control period.

Table 3 shows microbial community composition as assessed via 16S-targeted Illumina Sequencing at Family Level. Abundance (%) at microbial family level in the ascending (AC), transverse (TC) and descending colon (DC) reactors during the control (C) and two final treatment (TR) weeks upon supplementation of the probiotic formulation for three different donors tested (n=2). Statistical differences between control and treatment (C vs TR), as calculated with a 2-sided Student T-test are indicated with an asterisk (*).

TABLE 3

| | | Donor 1 | | | | | | Donor 2 | |
|---|---|---|---|---|---|---|---|---|---|
| | | AC | | TC | | DC | | AC | |
| | | C | TR | C | TR | C | TR | C | TR |
| Actinobacteria | Bifidobacteriaceae | 0.72% | 0.81% | 1.01% | 1.99% | 1.14% | 1.91% | 0.08% | 0.87% |
| | Microbacteriaceae | — | 0.38% | — | 0.19%(*) | — | 0.15%(*) | — | 0.44% |
| Bacteroidetes | Bacteroidaceae | 61.2% | 49.8% | 56.2% | 51.9% | 50.1% | 52.9% | 69.0% | 58.1% |
| | Porphyromonadaceae | — | 0.04% | 4.29% | 3.45% | 3.91% | 4.08% | 0.37% | 0.35% |
| | Prevotellaceae | — | — | — | — | — | — | — | — |
| | Rikenellaceae | — | — | 0.12% | 0.25% | 0.24% | 0.42% | — | — |
| Firmicutes | Acidaminococcaceae | — | — | — | — | — | — | — | 10.8%(*) |
| | Bacillaceae | — | 0.10% | — | 0.08% | — | 0.08%(*) | — | 0.09%(*) |
| | Clostridiaceae_1 | — | — | — | — | — | — | 5.20% | 3.60% |
| | Enterococcaceae | 0.03% | 0.19% | — | 0.07% | — | 0.04% | 0.03% | 0.11% |
| | Lachnospiraceae | 21.6% | 25.7% | 26.4% | 27.1% | 31.6% | 25.4% | 12.1% | 9.13% |
| | Ruminococcaceae | — | — | 0.52% | 3.15%(*) | 0.95% | 4.90%(*) | — | — |
| | Veillonellaceae | 12.9% | 18.6% | 4.25% | 4.23% | 2.62% | 1.71% | 9.42% | 11.5% |
| Fusobacteria | Fusobacteriaceae | — | — | 0.28% | 2.21% | 0.15% | 1.13% | — | — |
| Proteobacteria | Alcaligenaceae | 0.65% | 0.48% | 1.31% | 0.94% | 1.62% | 1.57% | — | 0.56% |
| | Brucellaceae | 0.08% | 0.35% | 0.04% | 0.35% | 0.03% | 0.18% | 0.20% | 0.08% |
| | Desulfovibrionaceae | — | 0.40% | 1.08% | 0.77% | 2.22% | 1.40% | — | 0.01% |
| | Enterobacteriaceae | 2.58% | 2.91% | 1.05% | 0.70% | 0.64% | 0.67% | 3.11% | 3.54% |
| | Pseudomonadaceae | 0.08% | 0.17% | 1.14% | 1.97% | 3.42% | 2.39% | 0.42% | 0.34% |
| | Rhodospirillaceae | — | — | 2.17% | 0.37% | 0.97% | 0.29% | — | — |
| | Xanthomonadaceae | 0.12% | 0.10% | 0.01% | 0.04% | — | 0.05% | 0.06% | 0.37% |
| Synergistetes | Synergistaceae | — | — | — | — | — | — | — | — |
| Verrucomicrobia | Verrucomicrobiaceae | — | — | — | — | — | — | — | — |

| | | Donor 2 | | | | Donor 3 | | | |
|---|---|---|---|---|---|---|---|---|---|
| | | TC | | DC | | AC | | TC | |
| | | C | TR | C | TR | C | TR | C | TR |
| Actinobacteria | Bifidobacteriaceae | 0.62% | 0.75% | 0.36% | 0.50% | 0.30% | 0.76%(*) | 0.25% | 0.40% |
| | Microbacteriaceae | — | 0.37% | — | 0.16% | — | — | — | — |
| Bacteroidetes | Bacteroidaceae | 53.7% | 33.1% | 51.2% | 44.4% | 23.0% | 23.4% | 32.8% | 29.3% |
| | Porphyromonadaceae | 9.71% | 10.5% | 9.20% | 4.33% | 1.23% | 1.25% | 1.97% | 4.75%(*) |
| | Prevotellaceae | — | — | — | — | 1.53% | 0.29% | 0.29% | 0.08% |
| | Rikenellaceae | 0.25% | 1.37%(*) | 0.24% | 0.89% | — | 0.01% | 0.01% | 0.04% |
| Firmicutes | Acidaminococcaceae | 2.82% | 7.77% | 2.94% | 11.8% | — | — | — | 0.16% |
| | Bacillaceae | — | 0.06% | — | 0.02% | — | — | — | — |
| | Clostridiaceae_1 | — | 0.05% | — | — | — | — | — | — |
| | Enterococcaceae | — | 0.02% | — | — | — | — | — | — |
| | Lachnospiraceae | 20.9% | 26.5% | 16.7% | 19.0% | 4.22% | 2.18%(*) | 5.75% | 8.89% |
| | Ruminococcaceae | 0.42% | 2.29%(*) | 0.41% | 3.01%(*) | — | — | 0.09% | 0.20% |
| | Veillonellaceae | 3.11% | 3.63% | 1.37% | 1.13% | 69.4% | 71.6% | 58.3% | 54.7% |
| Fusobacteria | Fusobacteriaceae | 0.30% | 0.14% | 0.14% | 0.07% | — | — | — | — |
| Proteobacteria | Alcaligenaceae | 0.70% | 0.89% | 0.86% | 1.16% | — | — | 0.06% | 0.01%(*) |
| | Brucellaceae | 0.04% | 0.07% | 0.04% | 0.05% | — | — | — | — |
| | Desulfovibrionaceae | 1.80% | 1.95% | 1.52% | 1.66% | 0.01% | — | 0.12% | 0.14% |
| | Enterobacteriaceae | 1.04% | 0.85% | 0.94% | 0.21% | 0.11% | 0.42% | 0.06% | 0.28% |
| | Pseudomonadaceae | 0.83% | 0.86% | 1.90% | 1.12% | 0.08% | 0.09% | 0.15% | 0.31%(*) |
| | Rhodospirillaceae | — | — | — | — | — | — | — | — |
| | Xanthomonadaceae | 0.02% | 0.01% | 0.01% | 0.04%(*) | — | — | — | — |
| Synergistetes | Synergistaceae | 3.60% | 8.09%(*) | 11.6% | 9.51% | — | — | — | — |
| Verrucomicrobia | Verrucomicrobiaceae | 0.08% | 0.58% | 0.39% | 0.77% | — | — | 0.17% | 0.76% |

| | | Donor 3 | |
|---|---|---|---|
| | | DC | |
| | | C | TR |
| Actinobacteria | Bifidobacteriaceae | 0.59% | 0.84% |
| | Microbacteriaceae | — | — |
| Bacteroidetes | Bacteroidaceae | 32.9% | 31.0% |
| | Porphyromonadaceae | 2.40% | 5.25% |
| | Prevotellaceae | 0.10% | 0.04% |
| | Rikenellaceae | 0.10% | 0.22% |
| Firmicutes | Acidaminococcaceae | — | 0.31%(*) |
| | Bacillaceae | — | — |
| | Clostridiaceae_1 | — | — |
| | Enterococcaceae | — | — |
| | Lachnospiraceae | 12.0% | 15.7% |
| | Ruminococcaceae | 0.24% | 0.40% |
| | Veillonellaceae | 48.4% | 41.4%(*) |
| Fusobacteria | Fusobacteriaceae | — | — |
| Proteobacteria | Alcaligenaceae | 0.14% | 0.06% |
| | Brucellaceae | — | — |
| | Desulfovibrionaceae | 0.34% | 0.30% |
| | Enterobacteriaceae | 0.03% | 0.59% |
| | Pseudomonadaceae | 1.67% | 2.56% |
| | Rhodospirillaceae | — | — |
| | Xanthomonadaceae | — | — |
| Synergistetes | Synergistaceae | — | — |
| Verrucomicrobia | Verrucomicrobiaceae | 0.97% | 1.32% |

(*)Statistical differences between control and treatment (C vs TR).

Finally, distinct differences in lactate levels were observed upon supplementation of the test product to the microbiota of the different donors (See, Table 2). Upon dosing the test product to donor 1, significantly increased lactate levels were observed in all colon regions. On the other hand, lactate levels remained unaffected upon dosing the probiotic formulation to donor 2, while for donor 3 even decreased lactate levels were observed, however only becoming statistically significant in the TC (p=0.010).

Analysis of the Microbial Community Composition

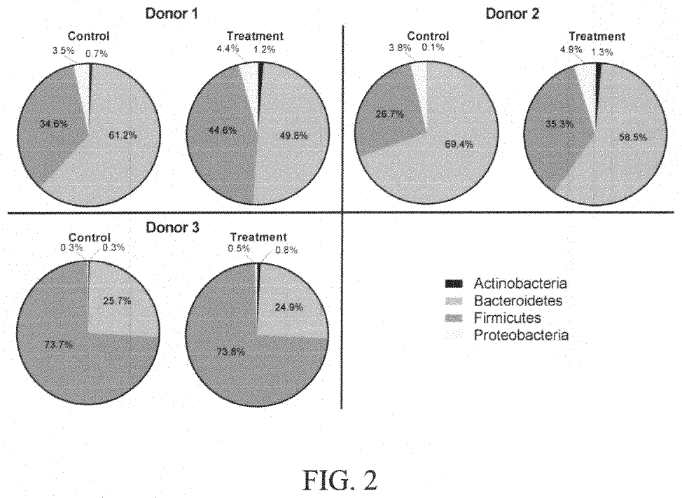

When comparing the samples at phylum level (FIG. 2; Table 3A), it followed that the main phyla in the microbial community of the three donors included Actinobacteria, Bacteroidetes, Firmicutes and Proteobacteria. Moreover, few sequences belonging to Fusobacteria were detected for donor 1 and 2, while donor 2 and 3 were characterized by low abundance of Verrucomicrobia. Finally, the microbial community of donor 2 also contained species belonging to the Synergistetes phylum, which specifically colonized the distal colon regions (TC and DC). Firmicutes and Bacteroidetes composed the largest fraction of the microbial community of the three donors. Furthermore, a colon-region specific colonization was observed as was shown by the Reciprocal Simpson Diversity Index which increased from AC to the distal colon regions TC and DC (See, Table 3A). Verrucomicrobia and Fusobacteria specifically colonized the distal colon areas.

Table 3A shows phylum composition as assessed via 16S-targeted Illumina Sequencing. Average Reciprocal Simpson Diversity Index together with abundances (%) of bacterial phyla in the ascending (AC), transverse (TC) and descending colon (DC) reactors during the control (C) and two final treatment (TR) weeks upon supplementation of the probiotic formulation for three different donors tested (n=2). Statistical differences between control and treatment (C vs TR), as calculated with a 2-sided Student T-test are indicated by the respective p-values. P-values<0.05 are indicated with an asterisk (*). Cf. FIG. 2.

TABLE 3A

| | | | AC | | TC | | DC | | CvsTR | | |
|---|---|---|---|---|---|---|---|---|---|---|---|
| | | | C | TR | C | TR | C | TR | AC | TC | DC |
| Donor 1 | Reciprocal Simpson Diversity Index | | 2.61 | 5.28 | 8.09 | 11.18 | 8.12 | 9.06 | 0.018(*) | 0.157 | 0.220 |
| | Phylum | Actinobacteria | 0.01 | 0.01 | 0.01 | 0.02 | 0.01 | 0.02 | 0.498 | 0.099 | 0.194 |
| | | Bacteroidetes | 0.61 | 0.50 | 0.61 | 0.56 | 0.54 | 0.57 | 0.094 | 0.072 | 0.489 |
| | | Firmicutes | 0.35 | 0.45 | 0.31 | 0.35 | 0.35 | 0.33 | 0.053 | 0.103 | 0.585 |
| | | Fusobacteria | 0.00 | 0.00 | 0.00 | 0.02 | 0.00 | 0.01 | — | 0.013(*) | 0.059 |
| | | Proteobacteria | 0.04 | 0.04 | 0.07 | 0.05 | 0.09 | 0.07 | 0.690 | 0.024(*) | 0.230 |
| Donor 2 | Reciprocal Simpson Diversity Index | | 2.99 | 4.72 | 6.65 | 12.40 | 9.39 | 9.49 | 0.267 | 0.111 | 0.979 |
| | Phylum | Actinobacteria | 0.00 | 0.01 | 0.01 | 0.01 | 0.00 | 0.01 | 0.006(*) | 0.374 | 0.178 |
| | | Bacteroidetes | 0.69 | 0.58 | 0.64 | 0.45 | 0.61 | 0.50 | 0.078 | 0.084 | 0.089 |
| | | Firmicutes | 0.27 | 0.35 | 0.27 | 0.40 | 0.22 | 0.35 | 0.171 | 0.152 | 0.041(*) |
| | | Fusobacteria | 0.00 | 0.00 | 0.00 | 0.00 | 0.00 | 0.00 | — | 0.150 | 0.370 |
| | | Proteobacteria | 0.04 | 0.05 | 0.04 | 0.05 | 0.05 | 0.04 | 0.704 | 0.861 | 0.388 |
| | | Synergistetes | 0.00 | 0.00 | 0.04 | 0.08 | 0.12 | 0.10 | 0.423 | 0.016(*) | 0.593 |
| | | Verrucomicrobia | 0.00 | 0.00 | 0.00 | 0.01 | 0.00 | 0.01 | — | 0.126 | 0.593 |
| Donor 3 | Reciprocal Simpson Diversity Index | | 2.04 | 1.92 | 2.46 | 2.95 | 3.25 | 4.35 | 0.783 | 0.217 | 0.030(*) |
| | Phylum | Actinobacteria | 0.00 | 0.01 | 0.00 | 0.00 | 0.01 | 0.01 | 0.021(*) | 0.469 | 0.478 |
| | | Bacteroidetes | 0.26 | 0.25 | 0.35 | 0.34 | 0.36 | 0.36 | 0.932 | 0.820 | 0.858 |
| | | Firmicutes | 0.74 | 0.74 | 0.64 | 0.64 | 0.61 | 0.58 | 0.990 | 0.957 | 0.517 |
| | | Proteobacteria | 0.00 | 0.01 | 0.00 | 0.01 | 0.02 | 0.04 | 0.723 | 0.255 | 0.345 |
| | | Verrucomicrobia | 0.00 | 0.00 | 0.00 | 0.01 | 0.01 | 0.01 | — | 0.091 | 0.225 |

(*)Statistical differences between control and treatment (C vs TR).

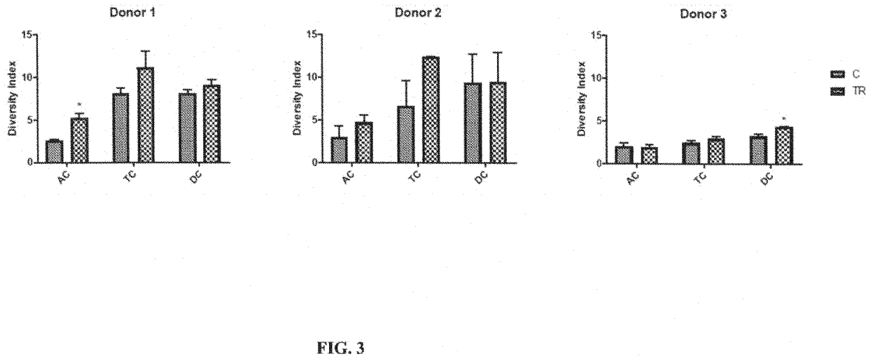

Upon treatment with the probiotic formulation, a consistent increase in the Actinobacteria phylum was observed in the AC for all three donors tested (FIG. 2). At family level this increase was mainly attributed to an increased abundance of Bifidobacteriaceae (Table 3). When further analysing data at lowest phylogenetic level (OTU level) within the Bifidobacteriaceae family, it followed that two OTUs were mainly responsible for the observed increases. These two OTUs were related to Bifidobacterium adolescentis and Bifidobacterium bifidum. Furthermore, the probiotic formulation tended to increase the diversity of the microbial communities of all donors in the distal colon (FIG. 3). Indeed, a tendency to increased diversity was observed in the TC for donor 1 and 2, with an increase of the Reciprocal Simpson Diversity Index from 6.65 to 12.40 for donor 1 (p=0.157) and from 8.09 to 11.18 for donor 2 (p=0.111). For donor 3 on the other hand, a significantly increased microbial diversity was observed in the DC (p=0.030). A wide-spectrum of groups containing propionate-producing species were found to increase in a donor-dependent fashion in the distal colon, especially for donor 2 and 3 (Table 3). For both donors, a tendency to increased Verrucomicrobiaceae was observed in the TC. Acidaminococcaceae tended to increase in all colon areas (p<0·100) for donor 2 and increased from below to above detection limit in both TC (p=0.129) and DC (p=0.009). Porphyromonadaceae increased both in TC (p=0.033) and DC (p=0·106) upon probiotic supplementation for donor 3, whereas only a slight increase was observed in the TC for donor 2. At OTU level, the change in this family was found to be mainly related to the increase of an OTU related to Parabacteroides distasonis for donor 3. Furthermore, for donor 2 a significant increase in the Rikenellaceae family was observed in the TC (p=0.013), which was mainly attributed to an increase in an OTU related to Alistipes onderdonkii. The probiotic formulation also indirectly favoured the presence of members belonging to the butyrate-producing families Lachnospiraceae and Ruminococcaceae in the distal colon areas for all donors tested (Table 3). However, within the latter family, OTUs related to different bacterial species were stimulated depending on the donor, i.e. Faecalibacterium prausnitzii for donor 1 and Subdoligranulum species for donor 2. Finally, the Verrucomicrobia reported data on the Verrucomicrobiaceae (only present in donor 2 and 3) where a tendency to increased Verrucomicrobiaceae was observed in the distal colon, especially in the TC (p=0.126 for donor 1 and p=0.091 for donor 3).

To assess the effect of the test product on specific taxonomic groups of interest (Faecalibacterium prausnitzii and Akkermansia muciniphila), qPCR analysis was performed (Table 4). A significant increase of Akkermansia muciniphila was observed in the AC only for donor 3 (from below the quantification limit to 5.4 log/mL). In the TC and DC, Akkermansia muciniphila colonized at much higher levels as compared to the AC, where an additional slight increase was observed in the TC of donor 3, though not statistically significant. Additionally, significantly increased levels of Faecalibacterium prausnitzii were observed in the distal colon areas (TC and DC) of donor 1. For donor 2, a similar trend was observed, though not statistically significant, whereas significantly reduced Faecalibacterium prausnitzii levels were observed for donor 3.

Table 4 shows microbial community composition as assessed via qPCR. Average Faecalibacterium prausnitzii and Akkermansia muciniphila levels (log 10 16S rRNA copies/mL) over the control (C; n=6) and the treatment (TR; n=9) period in the ascending (AC); transverse (TC) and descending colon (DC) of the human gastro-intestinal tract for three donors upon treatment with the probiotic formulation. Data is presented as mean±stdev. Statistically significant differences relative to the control period are indicated with an asterisk (p<0.05) (*).

TABLE 4

| | | Donor 1 | | Donor 2 | | Donor 3 | |
|---|---|---|---|---|---|---|---|
| | | C | TR | C | TR | C | TR |
| Faecalibacterium | AC | below | below | below | below | below | below |
| prausnitzii | | LOQ | LOQ | LOQ | LOQ | LOQ | LOQ |
| | TC | 6.3 | 7.4(*) | 7.7 | 8.2 | 8.3 | 7.7(*) |
| | | 0.0 | 0.3 | 0.5 | 0.4 | 0.2 | 0.3 |
| | DC | 5.5 | 7.4(*) | 7.2 | 8.1 | 8.3 | 7.9(*) |
| | | 0.2 | 0.5 | 0.1 | 0.7 | 0.2 | 0.1 |
| Akkermansia | AC | below | below | below | below | below | 5.4(*) |
| muciniphila | | LOQ | LOQ | LOQ | LOQ | LOQ | 0.1 |
| | TC | below | below | 7.7 | 8.1 | 8.4 | 8.4 |
| | | LOQ | LOQ | 0.5 | 0.1 | 0.2 | 0.2 |
| | DC | below | below | 7.9 | 7.9 | 8.6 | 8.7 |
| | | LOQ | LOQ | 0.1 | 0.2 | 0.1 | 0.1 |

(*)Statistically significant relative to control period.

By using a validated in vitro gut model, the long-term effect of a spore-based probiotic formulation (containing five different Bacillus strains) on the microbial metabolic activity and community composition in the human gastro-intestinal tract was assessed. To culture the complex gut microbial community under representative conditions, in vitro gut models depend on fecal samples from human individuals (Kaur, A., et al., “In vitro batch fecal fermentation comparison of gas and short-chain fatty acid production using “slowly fermentable” dietary fibers,” Journal of Food Science (2011) 76: H137-42). However, the human gut microbiome is characterized by large inter-individual differences, which can be affected by several factors such as age, sex, dietary habits, environmental and genetic aspects (Eckburg, P. B., et al., “Diversity of the Human Intestinal Microbial Flora,” Science (2005) 308: 1635-1638). As these differences can affect the response to probiotic supplementation, inter-individual variability must be taken into account in in vitro studies. The present study revealed a donor-dependent modulation of microbial metabolism and composition, with the main effects being observed in the distal colon.

Supplementation of the probiotic formulation significantly led to colonic acidification in the distal colon for donor 2 and 3, while no effect was observed for donor 1. These findings indicate that effects on the microbial community by repeated intake of the test product were donor-dependent, which was confirmed by the SCFA profiles. It followed that the increase in base consumption was mainly caused by significantly increased levels of acetate and propionate. Whereas donor 2 more specifically increased acetate concentrations, donor 3 was mainly characterized by increased propionate levels in the distal colon. At the same time, lactate levels tended to decrease in the AC for both donors, reaching significance in the TC of donor 3, which could be linked with the increased propionate concentrations. Indeed, lactate can be converted to the health-related SCFA propionate by the action of lactate-utilizing, propionate-producing microorganisms, such as Clostridium propionicum from the Lachnospiraceae family (Reichardt, N., et al., “Phylogenetic distribution of three pathways for propionate production within the human gut microbiota,” The ISME Journal (2014) 8: 1323-35). The health promoting activity of propionate is related to positive effects on glycemic control (Wong, J. M., et al., “Colonic health: fermentation and short chain fatty acids,” Journal of Clinical Gastroenterology (2006) 40: 235-43) and the inhibition of fatty acid and cholesterol synthesis in the liver (Demigne, C., et al., “Effect of propionate on fatty acid and cholesterol synthesis and on acetate metabolism in isolated rat hepatocytes,” The British Journal of Nutrition (1995) 74: 209-19). Donor 1 showed a different metabolic profile, as repeated intake of the probiotic formulation resulted in increased butyrate over propionate levels in the distal colon regions. Butyrate is considered as one of the main energy sources for the intestinal epithelial cells and has shown protective effects against inflammation and the development of colon cancer. In addition, butyrate has been linked with promotion of satiety and reduction of oxidative stress (Hamer, H. M, et al., “Review article: the role of butyrate on colonic function,” Alimentary Pharmacology & Therapeutics (2008) 27: 104-19).

Results on metabolic activity indicated that the probiotic formulation was able to alter the microbial metabolism, by either a direct increase in microbial activity by the probiotic strains and/or by an indirect effect of the test product in stimulating the growth of specific species in the microbial community of the different donors. The latter could be confirmed by 16S-targeted Illumina sequencing. For instance, the increased propionate production in the distal colon of donor 2 and 3 was associated with a donor-dependent stimulation of a wide spectrum of propionate-producing species, including Verrucomicrobiaceae and Acidaminococcaceae in both donors, Porphyromonadaceae in donor 3 and Rikenellaceae in donor 2. Furthermore, in agreement with increased acetate levels in the AC, Bifidobacteriaceae were found to increase for all three donors tested. Particularly two OTUs, related to Bifidobacterium adolescentis and Bifidobacterium bifidum, increased upon supplementation of the probiotic formulation. It has been reported that modulation of the gut microbiota could impact metabolic endotoxemia and that elevated colonic Bifidobacterium levels are positively correlated with decreased endotoxemia in high-fat diet-fed mice (Cani, et al., 2007). Furthermore, McFarlin, et al. (2017) reported that the same probiotic formulation as used during the present study reduced post-prandial metabolic endotoxemia in human subjects. The observed increase in Bifidobacterium spp. levels in the current study might therefore provide a possible mode-of-action of the spore-based probiotic formulation on metabolic endotoxemia in humans. The probiotic formulation also indirectly favoured the presence of members belonging to the butyrate-producing families Lachnospiraceae and Ruminococcaceae in the distal colon for all donors tested. Within the latter phylum, increased Faecalibacterium prausnitzii levels were observed for donor 1 and 2. Faecalibacterium prausnitzii has been shown to exert strong anti-inflammatory properties by the induction of regulatory T-cells and the production of the health-related metabolite butyrate (Furusawa, Y., et al., “Commensal microbe-derived butyrate induces the differentiation of colonic regulatory T cells,” Nature (2013) 504: 446-50), and it has been associated with the reduction of inflammatory markers in obese subjects. Furthermore, González-Sarrías, et al., (2018) reported a significant association between decreased endotoxemia and increased levels of Faecalibacterium. This further suggests that modulation of the gut microbiome by consumption of the spore-based probiotic supplement could be a possible mode-of-action in the reduction of post-prandial metabolic endotoxemia, shown by McFarlin, et al. (2017).

Moreover, by performing 16S-targeted Illumina sequencing, the colon-region specific colonization in the SHIME model was demonstrated with increasing microbial diversity from the AC to the distal colon regions TC and DC. This corresponds with the findings of Van den Abbeele et al. (Van den Abbeele, P., et al., “Microbial community development in a dynamic gut model is reproducible, colon region specific, and selective for Bacteroidetes and Clostridium cluster IX,” Appl. Environ. Microbiol. (2010) 76: 5237-46) and results from the imposed conditions in the AC that select for a specific carbohydrate fermenting microbial community, which is mainly composed of Firmicutes and Bacteroidetes. The distal colon was colonized by species that have previously been identified to thrive in distal areas based on specific metabolic functions to which they contribute. The latter include for instance Akkermansia muciniphila that specifically degrades mucins in the distal colon, leading to production of acetate and especially propionate (Van Herreweghen, F. et al., “In vitro colonisation of the distal colon by Akkermansia muciniphila is largely mucin and pH dependent,” Benef. Microbes (2017) 8: 81-96). Treatment with the probiotic formulation resulted in increased Akkermansia muciniphila levels for donor 3, which could (at least partially) explain the observed propiogenic effect (See, FIG. 1). Recently, it has been shown that Akkermansia muciniphila is capable of preventing adverse effects caused by high-fat diet-induced obesity, including fat-mass development, adipose tissue inflammation, insulin resistance and metabolic endotoxemia (Schneeberger, M., et al., “Akkermansia muciniphila inversely correlates with the onset of inflammation, altered adipose tissue metabolism and metabolic disorders during obesity in mice,” (2015) 5: 16643). It was reported by Everard, et al., “Cross-talk between Akkermansia muciniphila and intestinal epithelium controls diet-induced obesity,” Proceedings of the National Academy of Sciences (2013) 110: 9066-9071, that Akkermansia muciniphila plays a crucial role in restoration of the mucus layer in obesity-related disorders, thereby improving gut barrier function and contributing to the reduction of metabolic endotoxemia.

From this study, it can be concluded that the spore-based probiotic formulation, containing five different Bacillus strains, positively affects the human gut microbiome activity and composition, especially in the distal colon. Moreover, when compared with known literature, the generated data support a possible role of the exemplary formulation in protecting against obesity-related disorders, possibly by impacting metabolic endotoxemia. However, further clinical studies are warranted to study the efficacy of the spore-based probiotic formulation in tackling obesity-related disorders.

### Example A

Evaluation of the effect of a mix of 5 Bacillus strains in the Simulator of the Human Intestinal Microbial Ecosystem (SHIME®)

The aim of this project was to investigate the potential positive effects of one test product, i.e. MegaSporeBiotic, in the human gastrointestinal tract (GIT), making use of the SHIME® technology platform.

The main endpoints of the study were related to the effect of the product on activity and composition of the luminal gut microbiota, resulting in potential beneficial outcomes on the human host, as follows.

Activity of the gut microbiota: general markers for fermentation (acidification), and specific markers for saccharolytic fermentation (SCFA, lactate) or proteolysis (ammonium and branched SCFA) were measured.

Composition of the microbial community: Akkermansia muciniphila, Bacteroidetes, Firmicutes, Lactobacillus spp. and Bifidobacterium spp. were monitored and measured.

Composition of the microbial community was determined in great detail using 16S-targeted Illumina sequencing.

Gut barrier integrity (as assessed by measuring the TEER of a monolayer of Caco-2 cells).

Finally, LPS-induced cytokines production (pro- and anti-inflammatory) and NF-κB activity of Caco-2/THP-1Blue™ co-cultures were measured.

Within the frame of this project a SHIME® experiment was performed to assess the potential effect of one test product, i.e. MegaSporeBiotic (2 capsules/d, dosed at once), on the activity and composition of the luminal gut microbiome. The SHIME unit consisted of an ascending (AC), transverse (TC) and descending colon (DC). During a two-week control period, baseline values for microbial activity and composition were established. After the control period, a three-week treatment period was initiated, during which the test product was supplemented.

During the control period, SCFA levels (main marker for metabolic activity of the colonic microbiota) were very stable within (on average 91.1% similar between consecutive time points in control period) the SHIME unit. This indicated that the SHIME model was operated under its most optimal conditions resulting in stable colon microbiota. This stability is a prerequisite that any effects observed during the treatment truly result from the administered test products. Moreover, by performing 16S-targeted Illumina sequencing, the colon-region specific colonization in the SHIME® model was demonstrated with microbial diversity increasing from the AC over the TC to the DC. While the AC was colonized by carbohydrate-fermenting microbes, the distal colon was colonized by species that have previously been identified to thrive in distal areas based on specific metabolic functions to which they contribute. The latter include for instance Akkermansia muciniphila that is a specialist degrader of host-derived glycans and Proteobacteria which contain multiple protein-fermenting species.

The treatment with MegaSporeBiotic increased base consumption significantly in the distal colon (TC and DC) during the third week of treatment. This finding that MegaSporeBiotic only modulated the gut microbiota after repeated administration was confirmed when focussing on metabolic activity and microbial community composition.

With respect to metabolic activity, it followed that the distal increase in base consumption was mainly caused by significantly increased levels of propionate during the final two weeks of treatment in the TC and DC. Additionally, a trend towards higher acetate levels was observed in the AC, again during the final two weeks of treatment. At the same time, lactate levels decreased during the long-term MegaSporeBiotic treatment, especially in the final treatment week in the TC. Such a decrease in lactate levels can indicate both a decreased production or an increased consumption. Given the fact that metabolites of which lactate is a precursor (i.e. propionate), were increased upon MegaSporeBiotic treatment in the TC, it can be hypothesized that lactate consumption increased. Finally, MegaSporeBiotic significantly reduced ammonium levels in the distal colon (TC and DC), indicating a decreased proteolytic fermentation in the distal colon upon MegaSporeBiotic treatment. Considering that proteolytic fermentation has been associated with the production of toxic compounds and that the distal colon region is must vulnerable to colonic diseases, a reduction of the ammonium levels in this colon area can be seen as a beneficial effect of MegaSporeBiotic supplementation.

With respect to effects on microbial community composition as detected via quantitative PCR, several changes were observed in the ascending colon where MegaSporeBiotic increased the level of Bifidobacterium spp., which are beneficial saccharolytic bacteria capable of producing high concentrations of lactate (precursor of propionate and butyrate). Another beneficial modulation at community level in the ascending colon upon repeated MegaSporeBiotic supplementation included the significant increase in the propionate-producing, mucin-degrading Akkermansia muciniphila that has recently been shown to be capable of preventing adverse effects caused by a high-fat diet-induced obesity. Interestingly, in the TC and DC where Akkermansia muciniphila colonizes at much higher levels as compare to the AC, an increase was observed during the second and third week of treatment, thus coinciding with the increased levels of propionate in the TC and DC. Finally, significant increases in the Firmicutes:Bacteroidetes ratio were observed in both the AC and TC upon MegaSporeBiotic treatment.

By performing 16S-targeted Illumina sequencing, an even more detailed insight in the treatment effects on microbial community composition was obtained. This allowed to underpin aforementioned changes at metabolic level in even greater detail, as follows.

In agreement with the tendency to higher acetate levels in the AC, Bifidobacteriaceae were found to increase (particularly two OTUs related to B. adolescentis and B. bifidum).

MegaSporeBiotic increased the diversity of the microbial communities in the distal colon. In agreement with the increased levels of propionate in the distal colon, a wide spectrum of groups containing potent propionate producers were found to increase, including Verrucomicrobiaceae (=Akkermansia muciniphila), Acidaminococcaceae, Porphyromonadacaeae and multiple OTUs belonging to the Bacteroidaceae.

MegaSporeBiotic washed out Lachnospiraceae from the AC to the distal colon regions (TC and DC), while also the other family of potent butyrate producers, i.e. Ruminococcaceae, tended to increase in the DC. These shifts were likely not large enough to have caused changes at metabolic level.

The samples collected at the end of control and treatment periods were used to treat the Caco-2/THP1 co-cultures, in order to investigate the effect of the metabolites on gut-wall functioning in vitro. Firstly, MegaSporeBiotic did not show a significant protective effect on Caco-2 barrier function. However, MegaSporeBiotic showed some interesting immunomodulatory effects, which are marker specific. This product was found to have some immunosuppressing properties in vitro after fermentation, resulting in the decrease of some immune mediators, particularly chemoattractant proteins such as IL-8 (neutrophils recruiter) and MCP-1 (monocytes/macrophages recruiter). On the other hand, IL-10, a bona fide anti-inflammatory cytokine, did not increase, neither did IL-6, a cytokine involved in wound repair. In general, no pronounced differences were observed when comparing to the control SHIME samples. In particular, most changes were apparent upon treatment with TC samples, where differences from SHIME control reached significance only for the chemoattractant protein MCP-1.

As a final remark, it is speculated that the observed treatment effects could have been more pronounced by additionally triggering germination of the spores. It was observed that the capsules containing the Bacillus spores, were not easily breaking down during their passage in the simulated stomach. It is known from literature, that the primary signals that induce spore germination in the GIT are nutritional, but that in some Bacillus species low pH can activate the germination process. The (partly) absence of a pH shock in this specific experimental setup, could have limited the effect of some of the five administered Bacillus species.

Study Design.

The aim of this project was to assess the potential effect of 1 test product, containing a mix of 5 Bacillus strains, on the activity (as assessed via SCFA, lactate, branched SCFA and ammonia production) and composition (as assessed via qPCR and 16S-targeted Illumina sequencing) of the luminal gut microbiome. In order to deal with this research question, the SHIME® was used as this in vitro gut model allows to perform mechanistic studies in a well-controlled environment, thus limiting the interference of external factors.

Analysis of the Microbial Community Composition and Activity

An important (unique) characteristic of the SHIME is the possibility to work with a stabilized microbiota community and to regularly collect samples from the different intestinal regions for further analysis. The large volumes in the colonic regions allow to collect sufficient volumes of liquids each day, without disturbing the microbial community or endangering the rest of the experiment. The analysis for the current probiotic substrate includes its resulting microbial metabolites, and effects on the resident microbial community composition.

A number of microbial parameters are monitored throughout the entire experiment as part of the standard SHIME experiment. These measurements are necessary to evaluate the performance of the model and allow to monitor basic changes in the microbial community composition and activity due to the probiotic treatment.

Overall fermentative activity: Acid/base consumption: the production of microbial metabolites in the colon reactors alters the pH. Without continuous pH control (through the addition of acid or base), the pH would exceed the fixed intervals. Consumption of acid/base is continuously monitored during a SHIME experiment.

Microbial community activity (3×/week): (a) Short-chain fatty acids (SCFA): the concentrations of acetic acid, propionic acid and butyric acid were analyzed; (b) Lactate was measured; and (c) Ammonium and branched SCFA were measured (isobutyric acid, isovaleric acid and isocaproic acid) are markers of proteolytic fermentation, with rather adverse effects on host health.

Microbial community composition (1×/week): As part of the standard SHIME experiments, following groups were quantified in the lumen via qPCR: Akkermansia muciniphila; Bacteroidetes phylum; Firmicutes phylum; Lactobacillus spp.; and Bifidobacterium spp.

Further, microbial community composition during the SHIME experiment was also assessed via 16S-targeted Illumina sequencing (1×/week). 16S-based Illumina sequencing is a molecular technique which is also based on the amplification of the 16S rRNA gene. Because the Illumina sequencing method is PCR-based, microbial sequences are amplified till a saturation level is reached. Therefore, while information on a broad spectrum of (non-predefined) OTUs is obtained (>100 different of the most dominant OTUs), the results are presented as proportional values versus the total amount of sequences within each sample, thus providing semi-quantitative results. The methodology applied by ProDigest involves primers that span 2 hypervariable regions (V3-V4) of the 16S rDNA. Using a pair-end sequencing approach, sequencing of 2×250 bp results in 424 bp amplicons. Such fragments are taxonomically more useful as compared to smaller fragments that are taxonomically less informative. Besides processing the data at phylum and family level, specific OTUs that changed were identified, while also the Simpson diversity index was calculated as a measure of both diversity and evenness. The lowest possible value of the index is 1, representing a community consisting of only one OTU. The highest possible value is the total number of OTUs. The index will approach the maximal value more, when the OTU distribution is more even, while a community that is dominated by a small number of OTUs will result in values closer to 1. The higher the index, the larger the diversity and the larger the evenness.

Test Materials

One test product, MegaSporeBiotic, was tested in this project. MegaSporeBiotic was tested at a dose of 2 capsules/day. The mixture contains 5 different Bacillus strains, i.e. Bacillus indicus HU36, Bacillus clausii SC-109, Bacillus subtilis HU58, Bacillus licheniformis SL-307, and Bacillus coagulans SC-208.

I. Analysis of the Microbial Community Composition

Quantitative PCR (qPCR): qPCR is a molecular technique that is based on the quantification of specific bacterial sequences (16S rRNA genes) through amplification. The 16S rRNA gene consists of variable and conserved regions, spread over the gene. Due to their key role in protein expression, the conserved regions are characterized by very low evolutionary rates. Any mutations that occurred in these regions during evolution have inevitably led to the death of the corresponding organism. Conservation of these 16S rRNA gene sequences is thus responsible for their universal presence over the superkingdom Bacteria, and allows the design of universal primers targeting the complete bacterial pool in a sample. Next to these conserved gene regions, the 16S rRNA gene also contains nine variable regions (V1-V9), which are characterized by a much higher evolutionary rate. These gene regions are typically less essential for the survival of the organism, which is why any mutations in these regions did not lead to death of the organism during evolution. Considering their higher evolutionary rates, these gene regions are typically used to distinguish between different taxonomic groups of bacteria.

In summary, through careful selection of selective primers, qPCR allows the direct targeted quantification of taxonomic groups of interest in a microbial ecosystem. In the present study, qPCR was used to monitor Akkermansia muciniphila, Bifidobacterium spp., Lactobacillus spp., Bacteroidetes phylum and Firmicutes phylum.

As this technique is not dependent on the (lack of) culturability of bacteria, data generated with this methoqaund offer a reliable insight in the quantitative effects of the probiotic treatment on the microbial community.

A first group under investigation was Akkermansia muciniphila. This propionate-producing, mucin-degrading microbe has recently been shown to be capable of preventing adverse effects caused by a high-fat diet-induced obesity. Firstly, the data confirmed a recent finding that this species specifically colonizes distal colon regions (TC and DC), while virtually being absent in the AC. While Akkermansia levels were already high in TC and DC, MegaSporeBiotic further increased the levels in the TC during the second and third week of treatment. MegaSporeBiotic also significantly increased Akkermansia muciniphila levels in the AC toward the end of the treatment period, although the absolute levels remained very low so that the potential relevance of this result remains is questionable.

Lactobacilli and Bifidobacteria are regarded as beneficial saccharolytic bacteria. Both groups are capable of producing high concentrations of lactate. Lactate is an important metabolite in the human colon environment because of its antimicrobial properties, but also because it is the driver of a series of trophic interactions with other bacteria, resulting in the production of downstream metabolites. It followed that MegaSporeBiotic tended to decrease Lactobacilli levels in the distal colon (TC and DC) towards the end of the treatment period. On the other hand, Bifidobacteria levels significantly increased in the AC, mostly caused by an increase during the final two treatment weeks.

The phylum Bacteroidetes contains the most abundant propionate producers. Hence, in some cases a relationship can be found between propionate concentrations and the abundance of these organisms. However, Bacteroidetes levels were not affected by the treatment with MegaSporeBiotic. As a remark, the absence of clear a treatment effect on the Bacteroidetes phylum does not necessarily imply an absence of effect of all members of this phylum. As it includes many different species, it is possible that while some species specifically increased, others decreased, resulting in stable Bacteroidetes levels. Hence, it is still possible that several species of the Bacteroidetes phylum are responsible for the increased propionate production observed in the distal colon.

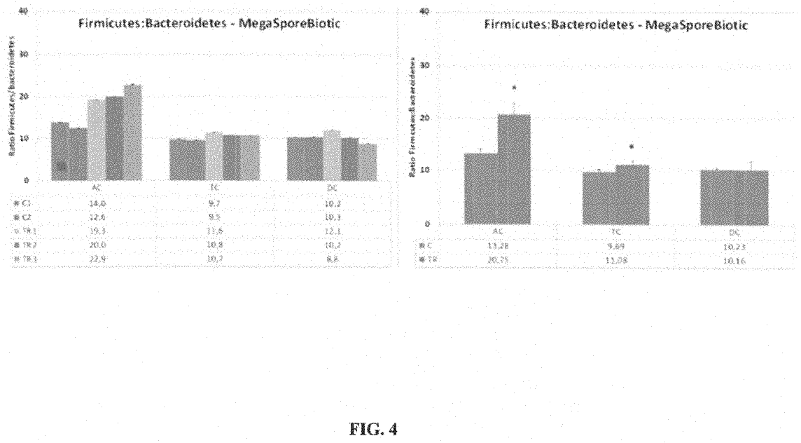

The phylum Firmicutes contains Clostridium clusters IV and XIVa, which are known to contain important propionate and butyrate producing organisms. An important class of propionate producers includes the Veillonellaceae (e.g. Veillonella and Megamonas sp.) that are potent lactate-consuming, propionate-producing members of the gut microbiome. Important groups of butyrate produces include the Ruminococcaceae (e.g. Faecalibacterium prausnitzii) and Lachnospiraceae (e.g. Roseburia). However, Firmicutes levels were not affected by the treatment with MegaSporeBiotic, which coincides with the absence of significant effects on butyrate production. On the other hand, significant increases in the Firmicutes:Bacteroidetes ratio were observed in both the AC (p<0.001) and TC (p=0.007) (FIG. 4). An increase in the Firmicutes:Bacteroidetes ratio has been previously reported upon prebiotic fiber intake, indicating its health-promoting potential. The effect of MegaSporeBiotic was most pronounced in the AC where a change with 8.9 (63.6%) was observed in favour of the Firmicutes phylum. This coincides with the trend towards higher butyrate concentrations that was observed in the AC.

16S-Targeted Illumina Sequencing

A. Colon-Region Specific Colonization

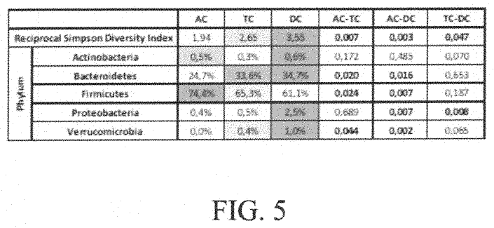

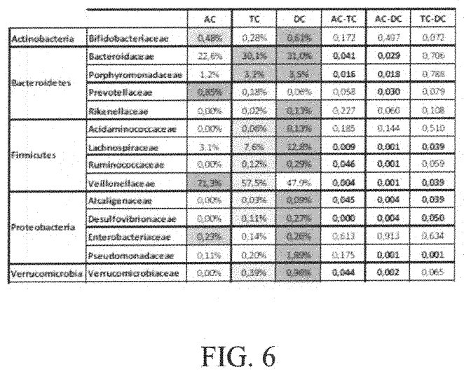

The differences in community composition between the AC, TC and DC region were characterized by processing the data both at phylum (FIG. 5) and family level (FIG. 6). Firstly, it followed that the Reciprocal Simpson Diversity Index increased from AC (1.94) over TC (2.65) to DC (3.55) (FIG. 5), corresponding to the findings of Van den Abbeele et al. (2010) 18. This can be explained by the fact that the imposed conditions in the ascending colon (lower pH, highest nutrient availability and higher bile salt concentration) select for a specific carbohydrate fermenting community, which was for the current donor under investigation mainly composed of Firmicutes (74%) and Bacteroidetes (25%) and a minor fraction of Actinobacteria (0.5%) and Proteobacteria (0.4%) (FIG. 5). Families that were specifically increased in the AC included the Prevotellaceae and Veillonellaceae. Given the fact that the SHIME system is a flow-through system, the AC community is physically transferred to the distal colon (TC and DC) where some members become inactive and where other microbial groups thrive based on specific metabolic functions to which they contribute in these distal colon regions, including results as follows.

1. Verrucomicrobiaceae of which Akkermansia muciniphila is the only representative in the gut microbiome. The current data confirm the recent finding that this species specifically degrades mucins in the distal colon, leading to the production of acetate and propionate.

2. Bacteroidaceae, Rikenellaceae and Porphyromonadaceae, containing many potent propionate producers (belonging to the Bacteroidetes).

3. Lachnospiraceae and Ruminococcaceae, containing many potent butyrate producers (belonging to the Firmicutes); as a remark, the increases in these families did not result in an overall increase of Firmicutes in the TC/DC given the high abundances of Veillonellaceae that specifically colonized the AC.

4. Alcaligenaceae, Desulfovibrionaceae and Pseudomonadaceae that belong to the Proteobacteria and are known to ferment proteins in distal colon regions.

B. Treatment Effects of MegaSporeBiotic

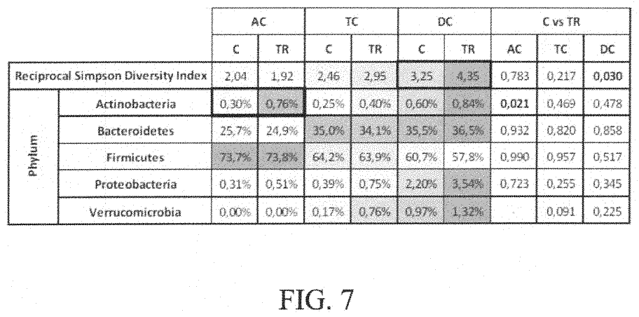

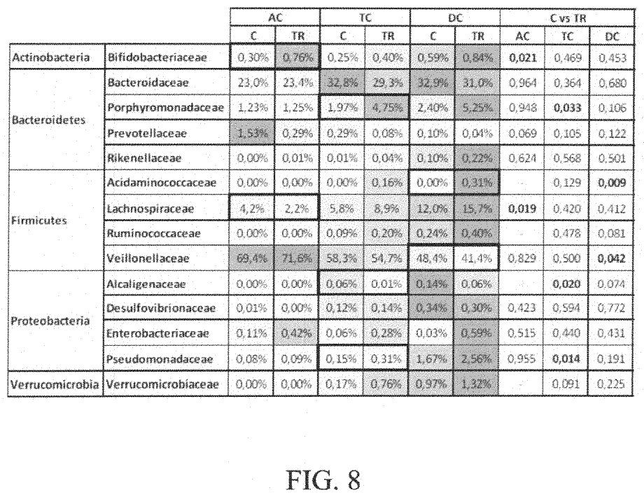

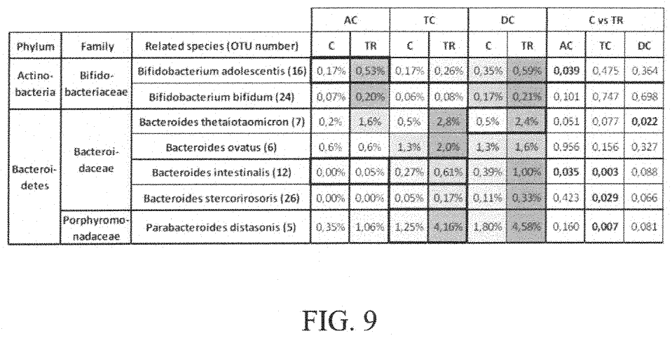

The treatment effects are again reported on different phylogenetic levels including the phylum (FIG. 7) and family level (FIG. 8). Moreover, based on changes reported at these levels, further investigation at the lowest possible level, i.e. OTU, level was performed (FIG. 9). To optimally focus on the treatment effects of MegaSporeBiotic, the Illumina data were processed by taking into account that at metabolic level, treatment effects of MegaSporeBiotic almost exclusively occurred during the second and third week of treatment. As a result, the average abundances of phyla, families and OTUs during the control period were compared to the averages during the final two weeks of treatment.

Firstly, it followed that MegaSporeBiotic increased the diversity of the microbial communities in the distal colon areas with a significant increase of the Reciprocal Simpson Diversity Index from 3.25 to 4.35 in the DC (p=0.03).

In terms of composition, it followed that in the AC, there was a significant increase in the Actinobacteria phylum (p=0.021), which was fully attributed to an increase in Bifidobacteriaceae at family level. At OTU level, the stimulation of Bifidobacteriaceae was caused by the increase of two Bifidobacterium OTUs, i.e. an OTU related to Bifidobacterium adolescentis (p=0.039) and one related to Bifidobacterium bifidum (p=0·101). The stimulation of Bifidobacteriacea likely resulted in the observed tendency to higher acetate levels in the AC during the final two treatment weeks. Further, the Bifidobacteriacea might have outcompeted Prevotellaceae that tended to decrease in the AC upon treatment with MegaSporeBiotic (p=0.069).

In agreement with the observations that MegaSporeBiotic increased propionate production and overall microbial diversity (FIG. 7) in the distal colon during the final two treatment weeks, it was found that MegaSporeBiotic treatment stimulated a wide spectrum of groups containing potential propionate-producing species in the distal colon at expense of Veillonellaceae that decreased tended to decrease in TC, while significantly decreasing in the DC (FIG. 8; p=0.042). The potential propionate-producing groups that were stimulated by MegaSporeBiotic including results as follows.

1. Verrucomicrobiaceae that tended to increase in the TC (p=0.091), an increase that was fully attributed to Akkermansia muciniphila, which is as mentioned above the only representative of this phylum in the human gut microbiome and known to produce priopionate upon degradation of host-derived glycans (mucins).

2. Acidaminococcaceae that increased from below to above detection limit in both TC (p=0.129) and DC (p=0.009). This family contains species such as Phascolarctobacterium faecium, that are known to convert succinate (metabolite of Bacteroidaceae species) to propionate

3. Porphyromonadacaeae that increased both in TC (p=0.033) and DC (p=0·106). At OTU level, the change in this family was found to be mainly related to the increase of an OTU related to Parabacteroides distasonis.

4. Several OTUs belonging to the Bacteroidaceae that also increased (even though the family as such did not increase) and were related to following species: B. thetaiotaomicron (TC—p=0.077; DC—p=0.022), B. ovatus (TC—p=0.156; DC—p=0.327), B. intestinalis (TC—p=0.003; DC—p=0.088) and B. stercorirosoris (TC—p=0.029; DC—p=0.066).

In contrast to what was expected based on decreases in ammonium production (FIG. 13), MegaSporeBiotic did not decrease the abundance of the Proteobacteria phylum. While the family of Alcaligenaceae did decrease (TC—p=0.020; DC—p=0.074), Pseudomonoadaceae increased in the TC (p=0.014) so that no direct links can be established between ammonium production and changes in Proteobacteria levels.

Finally, it seemed that the treatment with MegaSporeBiotic caused washout of Lachnospiraceae from the AC to the distal colon regions (TC and DC). Moreover, also the other family containing potent butyrate producers, i.e. Ruminococcaceae, tended to increase in the DC (p=0.081). As butyrate-producing species often depend on cross-feeding interactions with other microbes, MegaSporeBiotic might indirectly favor the presence of members belonging to the Lachnospiraceae and Ruminococcaceae.

Conclusions Re Microbial Community Composition

Firstly, as required during the control period, acid/base consumption, SCFA, lactate, ammonium and microbiota composition were all very stable within the SHIME unit, thus providing an excellent platform to assess effect of treatment with the test product, i.e. MegaSporeBiotic (2 capsules/d), a mixture of 5 Bacillus strains. During the control period, SCFA levels (main marker for metabolic activity of the colonic microbiota) were very stable within (on average 91.1% similar between consecutive time points in control period) the SHIME unit. This indicated that the SHIME model was operated under its most optimal conditions resulting in stable colon microbiota. This stability is a prerequisite that any effects observed during the treatment truly result from the administered test products. Moreover, by performing 16S-targeted Illumina sequencing, the colon-region specific colonization in the SHIME® model was demonstrated with microbial diversity increasing from the AC over the TC to the DC. While the AC was colonized by carbohydrate-fermenting microbes, the distal colon was colonized by species that have previously been identified to thrive in distal areas based on specific metabolic functions to which they contribute. The latter include for instance Akkermansia muciniphila that is a specialist degrader of host-derived glycans and Proteobacteria which contain multiple protein-fermenting species.

The treatment with MegaSporeBiotic increased base consumption significantly in the distal colon (TC and DC) during the third week of treatment. This finding that MegaSporeBiotic only modulated the gut microbiota after repeated administration was confirmed when focussing on metabolic activity and microbial community composition.

With respect to metabolic activity, it followed that the distal increase in base consumption was mainly caused by significantly increased levels of propionate during the final two weeks of treatment in the TC and DC. Additionally, a trend towards higher acetate levels was observed in the AC, again during the final two weeks of treatment. At the same time, lactate levels decreased during the long-term MegaSporeBiotic treatment, especially in the final treatment week in the TC. Such a decrease in lactate levels can indicate both a decreased production or an increased consumption. Given the fact that metabolites of which lactate is a precursor (i.e. propionate), were increased upon MegaSporeBiotic treatment in the TC, it can be hypothesized that lactate consumption increased. Finally, MegaSporeBiotic significantly reduced ammonium levels in the distal colon (TC and DC), indicating a decreased proteolytic fermentation in the distal colon upon MegaSporeBiotic treatment. Considering that proteolytic fermentation has been associated with the production of toxic compounds and that the distal colon region is must vulnerable to colonic diseases, a reduction of the ammonium levels in this colon area can be seen as a beneficial effect of MegaSporeBiotic supplementation.

With respect to effects on microbial community composition as detected via quantitative PCR, several changes were observed in the ascending colon where MegaSporeBiotic increased the level of Bifidobacterium spp., which are beneficial saccharolytic bacteria capable of producing high concentrations of lactate (precursor of propionate and butyrate). Another beneficial modulation at community level in the ascending colon upon repeated MegaSporeBiotic supplementation included the significant increase in the propionate-producing, mucin-degrading Akkermansia muciniphila that has recently been shown to be capable of preventing adverse effects caused by a high-fat diet-induced obesity. Interestingly, in the TC and DC where Akkermansia muciniphila colonizes at much higher levels as compare to the AC, an increase was observed during the second and third week of treatment, thus coinciding with the increased levels of propionate in the TC and DC. Finally, significant increases in the Firmicutes:Bacteroidetes ratio were observed in both the AC and TC upon MegaSporeBiotic treatment.

By performing 16S-targeted Illumina sequencing, an even more detailed insight in the treatment effects on microbial community composition was obtained. This allowed to underpin aforementioned changes at metabolic level in even greater detail, as follows.

In agreement with the tendency to higher acetate levels in the AC, Bifidobacteriaceae were found to increase (particularly two OTUs related to B. adolescentis and B. bifidum).

MegaSporeBiotic increased the diversity of the microbial communities in the distal colon. In agreement with the increased levels of propionate in the distal colon, a wide spectrum of groups containing potent propionate producers were found to increase, including Verrucomicrobiaceae (=Akkermansia muciniphila), Acidaminococcaceae, Porphyromonadacaeae and multiple OTUs belonging to the Bacteroidaceae.

MegaSporeBiotic washed out Lachnospiraceae from the AC to the distal colon regions (TC and DC), while also the other family of potent butyrate producers, i.e. Ruminococcaceae, tended to increase in the DC. These shifts were likely not large enough to have caused changes at metabolic level.

As a final remark, it is speculated that the observed treatment effects could have been more pronounced by additionally triggering germination of the spores. It was observed that the capsules containing the Bacillus spores, were not easily breaking down during their passage in the simulated stomach. It is known from literature, that the primary signals that induce spore germination in the GIT are nutritional, but that in some Bacillus species low pH can activate the germination process. The (partly) absence of a pH shock in this specific experimental setup, could have limited the effect of some of the five administered Bacillus species.

II. Host-Microbiome Interaction Study

Gut microbes form a biologically active community that lies at the interface of the host with its nutritional environment. As a consequence, they profoundly influence several aspects of host's physiology and metabolism. A wide range of microbial structural components and metabolites directly interact with host intestinal cells to influence nutrients uptake and epithelial health. Ultimately, both microbial associated molecular patterns (MAMPs) and bacterial-derived metabolites such as SCFA contribute to or trigger various signaling pathways, namely: lymphocyte maturation, epithelial health, neuroendocrine signaling, pattern recognition receptors (PRRs)-mediated signaling and G-protein coupled receptors (GPRs)-mediated signaling. In turn, these will dictate inflammatory tone, energy balance, gut motility and appetite regulation. Dysregulation of host-microbiome interactions is nowadays recognized as being at the onset and contribute to numerous diseases, including: metabolic syndrome and obesity, inflammatory bowel diseases (IBD) such as Crohn's disease (CD) and ulcerative colitis (UC), irritable bowel syndrome (IBS), celiac disease, diabetes, allergies, asthma and autoimmune diseases. Commonly to all these disorders, one of the first mechanisms that seems to be dysregulated and initiate pathology is an altered (more permeable) intestinal epithelial barrier. When intestinal barrier function is disrupted, the trafficking of molecules is no longer under control, and so luminal contents may enter the lamina propria and activate the immune system, thereby leading to uncontrolled immune responses (in a process known as “leaky gut”). The intestinal epithelial barrier, with its intercellular tight junctions, controls the equilibrium between immune tolerance and immune activation, and therefore has a prominent role in “leaky gut” pathogenesis. Thus, tight junctions form a complex protein-protein network that mechanically links adjacent cells and seals the intercellular space. Therefore, inadequate functioning or regulation of tight junctions at the level of the gut wall seems to be responsible for enlarged intercellular spaces with concomitant transport of luminal elements across the barrier and consecutive local and systemic inflammation.

Caco-2/THP1 Co-Culture In Vitro Model

In order to mimic the interface between host and gut microbiome, several in vitro models have been developed in the past years that include the use of intestinal epithelial-like cells and immune cells of human origin. At ProDigest, we make use of a co-culture of intestinal epithelial-like cells (Caco-2 cells) and human monocytes/macrophages (THP1 cells), in a model based on the work of Satsu and colleagues (Exp. Cell Res. (2006) 312:3909-3919). Caco-2 cells, when seeded on suitable supports, spontaneously differentiate into mature enterocyte-like cells, characterized by polarization, presence of villi, formation of domes, presence of tight junctions and vectorial transport and expression of apical brush-border enzymes.

THP1 monocytes, isolated from a human patient with acute leukemia, are able to differentiate into macrophage-like cells upon phorbol 12-myristate 13-acetate (PMA) treatment. PMA-activated THP1 cells acquire morphological features characteristic of macrophages, are able to adhere to the support, develop lamellipodia necessary for migration and phagocytosis and become primed for toll-like receptor (TLR) responses.

When Caco-2 cells are placed on top of PMA-activated THP1 cells, which secrete cytokines into the supernatant, their monolayer becomes disrupted and a decrease in transepithelial electrical resistance (TEER) is observed. This is possibly due to cytokine-mediated disruption of tight junctions. Therefore, TEER is a measure of barrier function and monolayer integrity.

Within the gut, chemical, mechanical or pathogen-triggered barrier disruption may lead to the influx of bacteria from the lumen into the lamina propria. This will activate the immune system, which will switch from a physiological “tolerogenic” inflammation into a detrimental pathological inflammation. An inflammatory signaling cascade will initiate with the production of alarm molecules such as pro-inflammatory cytokines (e.g. tumor necrosis factor (TNF)-α and interleukin (IL)-1β). TNF-α, together with interferon (IFN)-γ, is produced by leukocytes but also by CD4+ TH (helper) type 1 cells, critical cellular defenders against invading microorganisms.

These inflammatory cytokines will induce the production of chemokines (such as IL-8 and C-X-C motif chemokine (CXCL)-10) and adhesion molecules, which in turn will lead to the recruitment of neutrophils and to the production of reactive oxygen species (ROS). These are necessary to kill the bacteria and to plug possible breaches in the epithelial wall. However, they may also cause tissue disruption and lead to inflammation. Therefore, cytokines involved in the resolution of inflammation will be activated. Among these are IL-6 and IL-10.

IL-6, a cytokine with both pro- and anti-inflammatory properties, through activation of monocyte chemoattractant protein (MCP)-1, leads to monocytes/macrophages recruitment that promote clearance of neutrophils. IL-6 is also able to inhibit the production of pro-inflammatory cytokines such as IL-1. Moreover, IL-6 has a positive effect on the regeneration of the intestinal epithelium and wound healing. On the other hand, IL-6, together with transforming growth factor (TGF)-β, induces the differentiation of an important subset of CD4+ T cells—TH17 cells—that have a key role in host defence against extracellular microbes in mucosal tissues.

IL-10 is able to suppress several cells from both innate and adaptive immune systems, to induce activation of anti-inflammatory molecules and to enhance T regulatory cell (Treg) function which in turn, will restore immune homeostasis. When these switch-off mechanisms are impaired and immune homeostasis cannot be restored, gut pathology can occur and this may result in chronic inflammation (as seen for example in IBD, which is characterized by an overactivation of TH1-mediated responses, namely by overproduction of TNF-α).

In terms of inflammation, TNF-α is one of the most important and dangerous cytokines produced by the immune system as it has potent pleiotropic effects and is able to amplify inflammation. When not counteracted, TNF-α can lead to chronic inflammation and even death in cases of acute inflammation. For this reason, anti-TNF-α therapy is widely used in several chronic inflammatory conditions, including IBD and rheumatoid arthritis.

As suggested by Satsu and colleagues (2006) this model shows some features that are also observed in IBD patients, and therefore they suggest that this “IBD-like” model may be used for testing the effect of substances that can on one hand, protect intestinal epithelial barrier integrity (by maintaining or inducing an increase in TEER) and on the other hand, reduce inflammation (by reducing pro-inflammatory cytokines production and increasing anti-inflammatory cytokines).

In this experiment, the colonic suspensions collected from the SHIME (or others) are brought in contact with the apical side of the co-cultures (Caco-2 cells). The effects observed on the basolateral chamber (where the THP1 cells are) are then mediated indirectly by the signalling produced by the Caco-2 cells and/or by the transport of micro and macromolecules. The unique aspect of this approach resides in the fact that it allows evaluating the effect induced by the product and the fermentation-derived metabolites produced by the gut microbiota during the digestive steps (and not only by the pure product alone). For more insight into this model see also Daguet et al., J. Functional Foods (2016) 20: 369-379.

Study Design

The aim of this section of the study was to investigate the potential effect of MegaSporeBiotic on gut-wall functioning after colonic fermentation. Because bacteria closely interact with the gut-wall, modulation of microbial activity and abundance by probiotics is likely to affect gut-wall function. This will be assessed by evaluating intestinal epithelium barrier integrity and specific immune markers in vitro.

Materials and Methods

Samples collected from the SHIME experiment have been used to evaluate in vitro the effect of the fermented product on intestinal epithelial barrier function and immune markers. These include samples from the ascending, transverse and descending colon reactors collected at the end of the control and treatment period with MegaSporeBiotic.

Caco-2 cells: The co-culture experiment was performed as previously described (Daguet et al., 2016). Briefly, Caco-2 cells (HTB-37; American Type Culture Collection) were seeded in 24-well semi-permeable inserts (0.4 μm pore size) at a density of 1×105 cells/insert. Caco-2 cell monolayers were cultured for 14 days, with three medium changes/week, until a functional cell monolayer with a transepithelial electrical resistance (TEER) of more than 300 Ω·cm2 was obtained. Cells were maintained in Dulbecco's Modified Eagle Medium (DMEM) containing 25 mM glucose and 4 mM glutamine and supplemented with 10 mM HEPES and 20% (v/v) heat-inactivated (HI) fetal bovine serum (FBS).

THP-1 cells: THP1-Blue□ (InvivoGen) cells were maintained in Roswell Park Memorial Institute (RPMI)1640 medium containing 11 mM glucose and 2 mM glutamine and supplemented with 10 mM HEPES, 1 mM Sodium pyruvate and 10% (v/v) HI-FBS. THP1-Blue□ are THP1 human monocytes stably transfected with a reporter construct expressing a secreted alkaline phosphatase (SEAP) gene under the control of a promoter inducible by the transcription factor nuclear factor kappa B (NF-κB). Upon TLR activation by molecules such as LPS (lipopolysaccharides; isolated from Gram-negative bacteria), NF-κB becomes activated and induces the expression and secretion of SEAP. This is then measured in the supernatant by using the QUANTI-Blue reagent (InvivoGen). Shortly, THP1-Blue cells were seeded in 24-well plates at a density of 5×105 cells/well and treated with 100 ng/mL of PMA for 48 hours (h). PMA induces the differentiation of the cells into macrophage-like cells that able to adhere and are primed for TLR signaling.

Caco-2/THP1 co-cultures: Before co-culture, the TEER of the Caco-2 monolayers was measured by using an Epithelial Volt-Ohm meter (0 h time point) (Millicell ERS-2 from Millipore). The TEER of an empty insert was subtracted from all readings to account for the residual electrical resistance of an insert. Then, the Caco-2-bearing inserts were placed on top of the PMA-differentiated THP1-Blue cells for further experiments, using the standard method. Shortly, the apical compartment (containing the Caco-2 cells) was filled with sterile-filtered (0.22 μm) colonic SHIME suspensions (diluted 1:5 (v/v) in Caco-2 complete media). Cells were also treated apically with Sodium butyrate (NaB) (Sigma-Aldrich; 12 mM) as positive control. The basolateral compartment (containing the THP1-Blue cells) was filled with Caco-2 complete media. Cells were also exposed to Caco-2 complete media in both chambers as control. Cells were treated for 24 h, after which the TEER was measured (24 h time point). After subtracting the TEER of the empty insert, all 24 h values were normalized to its own 0 h value (to account for the differences in initial TEER of the different inserts) and are presented as percentage of initial value. Then, the basolateral supernatant was discarded and cells were stimulated basolaterally with Caco-2 complete media containing 500 ng/mL of ultrapure LPS (Escherichia coli K12, InvivoGen). Cells were also stimulated basolaterally with LPS and hydrocortisone (HC) (Sigma-Aldrich; 1 μM) and media without LPS (LPS−) as controls. After 6 h of LPS stimulation the basolateral supernatant was collected for cytokines measurement (human IL-1β, IL-6, IL-8, IL-10, TNF-α, CXCL10 and MCP-1 by Luminex® multiplex (Affymetrix-eBioscience)) and for assessing NF-κB activity, according to the manufacturers' instructions. All treatments were done in triplicate. Cells were incubated at 37° C. in a humidified atmosphere of air/CO2 (95:5, v/v).

Statistics

The experimental controls are presented first in separate plots; these relate to the complete media control (CM or LPS−), the lipopolysaccharide (LPS+)-treated cells and the Sodium butyrate (NaB) and hydrocortisone (HC) controls. Concerning the TEER, the conditions CM and NaB are compared and statistical significances were calculated by using unpaired, two-tailed Student's t-test. For the immune markers (cytokines/chemokines and NF-κB activity) all conditions are compared to LPS+ (LPS−, LPS+HC and LPS+NaB). Statistical significances were calculated by using one-way ANOVA with Dunnett's multiple comparisons test against LPS+. (*), (**), (***) and (****) represent p<0.05, p<0.01, p<0.001 and p<0.0001, respectively.

The results concerning the SHIME samples are presented separately. The control and treatment periods of the SHIME are presented for all colon reactors (ascending, transverse and descending colon). To evaluate the difference between the treatment sample and respective control sample, a two-way ANOVA with Sidak's multiple comparisons test was performed (significances are depicted with an asterisk (*)). (*), (**), (***) and (****) represent p<0.05, p<0.01, p<0.001 and p<0.0001, respectively. All statistics were performed using GraphPad Prism version 7.03 for Windows (GraphPad Software, San Diego, Calif., USA).

Due to the large amount of measurements, all mean values of SHIME treatment samples were normalized to the respective controls and presented in percentages. This normalization gives a better perspective of the changes in cytokine expression due to the treatment with MegaSporeBiotic.

Controls

Transepithelial Electrical Resistance (TEER)

After 24 h co-culture incubation, the complete media (CM) control shows approximately a 30% decrease in TEER due to the damage induced by the PMA-activated THP1 cells on Caco-2 cells (FIG. 24). As expected, Sodium butyrate (NaB; positive control) is able to protect Caco-2 cells from this damage, and to maintain the TEER of the monolayer. Note that LPS is only added after the TEER has been measured at 24 h. However, preliminary experiments have shown that the dose of LPS used does not significantly affect the barrier integrity of these cells.

Immune Markers

Various immune markers were measured. As expected, LPS is able to increase the secretion of all immune markers. In contrast, hydrocortisone (HC), being a corticosteroid, acts as a broad immunosuppressant by reducing LPS-induced cytokines and chemokines and also by inhibiting LPS-induced transcriptional activity of NF-κB. In contrast, Sodium butyrate (NaB) shows marker-dependent effects. Although NaB increases the transcriptional activity of NF-κB, an effect which is possibly mediated by the attenuation of histone deacetylase (HDAC) inhibitory activities on non-histone proteins such as NF-κB37,38, it also has clear selective post-transcriptional inhibitory activities on some immune mediators, such as CXCL10. Thus, in this experiment, NaB was shown to selectively increase LPS-induced IL-10 and IL-6 (involved in immune homeostasis) and to selectively inhibit LPS-induced TNF-α (pro-inflammatory cytokine) and CXCL10, IL-8 and MCP-1 (chemokines involved in recruitment of immune cells). The results obtained for the SHIME samples are presented in the next section.

SHIME Samples

Transepithelial Electrical Resistance (TEER)

The samples collected during the last weeks of control and treatment from all colon reactors were diluted (1:5, v/v) in Caco-2 complete media after filtration (0.22 μm) and were added to the apical side of the co-cultures for 24 h.

When compared to the complete media (CM) control, where a TEER decrease of approximately 30% was observed, all samples collected from the SHIME (including the controls) were able to maintain the TEER at the initial value, and so to protect the cells from the THP1-induced disruption on barrier function. When comparing the SHIME treatment samples to the respective controls, slightly higher TEER values were observed for the TC and slightly lower for the AC and DC samples, despite general lack of significance. Therefore, it was concluded that the fermentation-derived metabolites collected from the SHIME inoculated with MegaSporeBiotic had no major effect on Caco-2 cells barrier function.

Immune Markers

After 24 h of pre-treatment of the Caco-2/THP1-Blue co-cultures with SHIME samples, the basolateral supernatant was discarded and the cells stimulated with LPS. After 6 h of stimulation, the basolateral supernatant was collected to measure cytokines and chemokines and to determine NF-κB activity.

When compared to the LPS+ control, all samples increased LPS-induced NF-κB transcriptional activity. Concerning the treatment samples, when compared to the respective controls, no statistical significant differences were found.

Similar to the results obtained for NF-κB activity, all SHIME samples increased LPS-induced IL-6 and IL-10 levels. However, lower IL-6 and IL-10 levels were observed for all treatment samples comparatively to the respective controls. These never reached significance, except for IL-10 in AC.

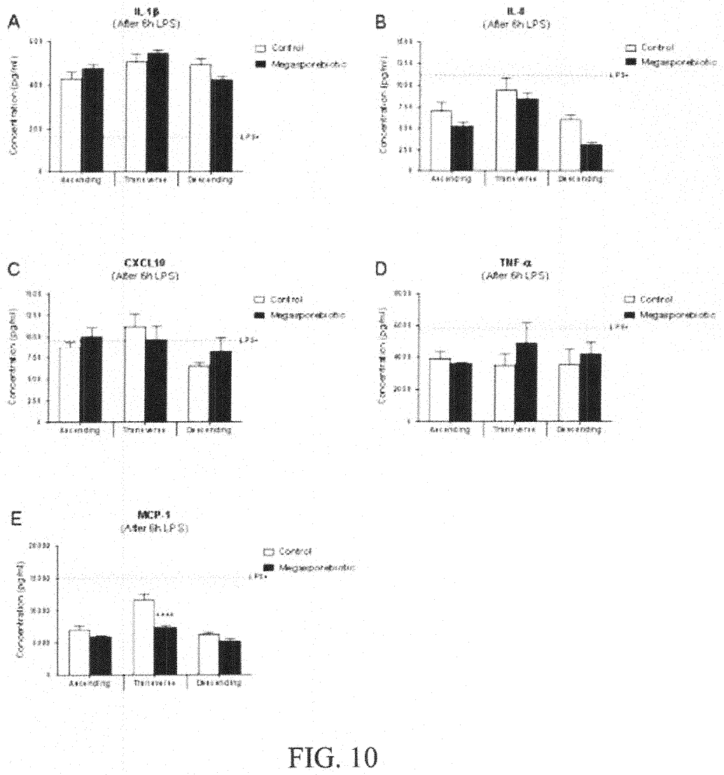

The results obtained for IL-1β, IL-8, CXCL10, TNF-α and MCP-1 are shown in FIG. 10. Whereas all SHIME samples, including the controls, increased LPS-induced IL-1β, all samples had inhibitory effects on LPS-induced IL-8, TNF-α and MCP-1 levels (see comparison to LPS+ depicted as a dotted line).

When comparing the treatment samples to the respective controls, IL-1β levels were found to be slightly higher upon treatment with samples collected from the AC and TC reactors, and to be slightly lower for the DC treated with MegaSporeBiotic. However, the levels never reached significance when compared to the controls (FIG. 10A).

Regarding IL-8, a decrease is observed for all colon departments. These never reached significance, despite a clear tendency to significance in the DC (p=0.0584) (FIG. 10B).

Slightly higher CXCL10 levels were observed in those cells treated with AC and DC MegaSporeBiotic samples. In contrast, slightly lower CXCL10 levels were obtained with the TC treated with MegaSporeBiotic (FIG. 10C), despite lack of significance.

Concerning TNF-α, lower levels are observed for the AC, whereas in contrast, an increase is seen in the TC and DC (FIG. 10D), despite lack of significance.

Finally, regarding MCP-1 levels (FIG. 10E), a decrease in this chemokine is observed for all samples treated with MegaSporeBiotic. Nevertheless, significance was only reached for the TC.

In general, MegaSporeBiotic treatment did not greatly affect LPS-induced NF-κB activity, IL1-β, CXCL10 or TNF-α, but did decrease the levels of IL-8 and MCP-1 in all colon departments in vitro.

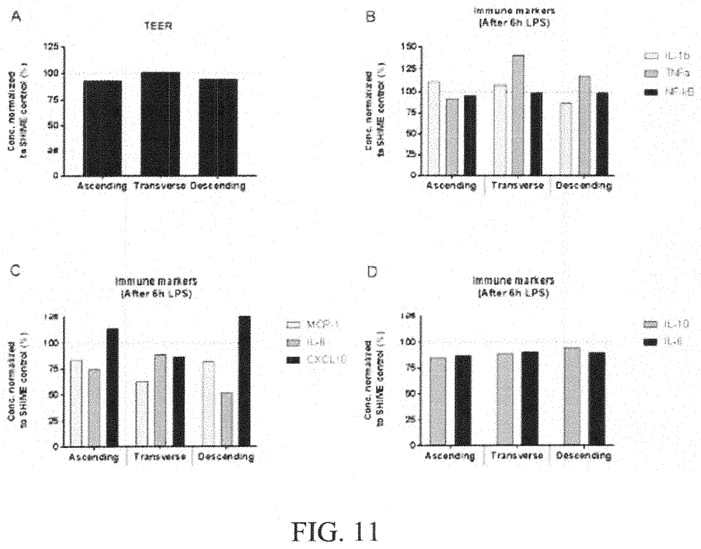

To have a better overview on the extent of changes induced by MegaSporeBiotic, all mean values of SHIME treatment samples were normalized to the respective controls and are presented as percentages. This normalization gives a clearer insight on the changes in cytokine expression due to the treatment with MegaSporeBiotic (FIG. 11), as it normalizes all control samples to 100%

MegaSporeBiotic treatment does not show a significant protective effect on Caco-2 barrier function (FIG. 11A). In contrast, it shows some interesting immunomodulatory effects, which are marker specific (FIG. 11B-C).

MegaSporeBiotic fermentation-derived metabolites are able to decrease the expression of pro-inflammatory cytokines such as MCP-1 and IL-8, who are chemokines responsible for recruitment of monocytes/macrophages and neutrophils respectively. Despite these observations, IL-1β, TNF-α, NF-κB and CXCL10 do not change in a clear and significant manner upon treatment, as they display colon compartment specific modulations (FIG. 11B-C). In addition, cytokines involved in immune resolution such as IL-6 and IL-10 slightly decrease when compared to their respective controls (FIG. 11D).

Conclusions re intestinal epithelial permeability and specific immune markers in vitro.

The aim of this study was to investigate the potential indirect effect of a Bacillus spore mix, MegaSporeBiotic, on gut-wall functioning. This was done by evaluating intestinal epithelial permeability and specific immune markers in vitro.

MegaSporeBiotic did not show a significant protective effect on Caco-2 barrier function.

However, MegaSporeBiotic showed some interesting immunomodulatory effects, which are marker specific. This product was found to have some immunosuppressing properties in vitro after fermentation, resulting in the decrease of some immune mediators, particularly chemoattractant proteins such as IL-8 (neutrophils recruiter) and MCP-1 (monocytes/macrophages recruiter).

On the other hand, IL-10, a bona fide anti-inflammatory cytokine, did not increase, neither did IL-6, a cytokine involved in wound repair.

In conclusion, the effects of MegaSporeBiotic are rather mild, and no pronounced differences are observed when comparing to the control SHIME samples. In particular, most changes are apparent upon treatment with TC samples, where differences from SHIME control reached significance only for the chemoattractant protein MCP-1.

### Example B

Evaluation of the effect of a Bacillus indicus strain HU36 in the Simulator of the Human Intestinal Microbial Ecosystem (SHIME®)

Denaturing Gradient Gel Electrophoresis (DGGE} creates a barcode from a microbial community, in which roughly on band in the barcode corresponds to one type of species. By creating such barcodes from samples collected at different time points during the SHIME experiment, and by comparing these profiles, qualitative changes in the microbiota over time (due to a specific treatment) can be evaluated.

The results from the experiment with HU36 were combined with the results from experiments with two other bacterial strains (GB1 and HU58). The principle of the analysis is that the similarity between the ‘barcodes’ from different samples is investigated. Samples with similar microbial community composition will group together, while samples with a different microbial community composition will be positioned separately from the other samples. In DGGE fingerprint, each horizontal barcode represents the microbiota composition of a sample collected from the 3 different SHIME experiments, during the control and treatment period.

The following conclusions can be drawn from the experiment.

Similarity analysis of the barcodes shows that the 2 samples from the control period (C1 and C2) from the HU36 experiment, i.e. before administration of the strain, group together with a similarity of about 85%.

Administration of HU36 induces a clear change in the microbiota composition. This is shown by the fact that the 2 samples from the treatment period (T1 and T2) group separately from the control samples, with a similarity of only 65%.

This confirms the previous observations that intake of strain HU36 has a profound effect on the intestinal environment, both in terms of microbiota community composition and activity.

The SHIME experiments provide a representative summary of the effects which can be expected upon repeated intake of HU36. The main conclusions are as follows.

HU36 was able to germinate under colonic conditions. Strain HU36 was able to multiply and resporulate, resulting in increased numbers of both vegetative cells and spores as compared to the administered dose.

HU36 affected the intestinal environment, as shown by increased levels of SCFA. Interestingly, butyrate was an important end product. Given the known effects of butyrate on the development of a healthy immune system and the prevention of colon cancer, these are important findings in relation to potential probiotic properties of the strain. The strain mainly affected saccharolytic fermentation, without influencing strongly proteolytic fermentation.

HU36 induced specific changes in the gut microbiota composition as indicated by specific changes in the DGGE community fingerprints.

### Example C

Evaluation of the effect of a Bacillus indicus strain HU58 in the Simulator of the Human Intestinal Microbial Ecosystem (SHIME®)

The experiment was performed as in Example B, and the following conclusions can be drawn.

Similarity analysis of the barcodes shows that the 2 samples from the control period (C1 and C2) from the HU-58 experiment, i.e. before administration of the strain, group together with a similarity of about 85%. This shows that the microbial community composition was therefore highly stable during the control period.

Administration of HU-58 induces a clear change in the microbiota composition. This is shown by the fact that the 2 samples from the treatment period (T1 and T2) group separately from the control samples, with a similarity of only 70%.

This confirms the previous observations that repeated intake of strain HU-58 has a profound effect on the intestinal environment, both in terms of microbiota community composition and activity.

The SHIME experiments provide a representative summary of the effects which can be expected upon repeated intake of HU58. The main conclusions are as follows.

HU58 was able to germinate under colonic conditions and could maintain itself in doses similar as the ingested dose.

HU58 affected the intestinal environment, as shown by increased levels of SCFA. Interestingly, butyrate was an important end product. Given the known effects of butyrate on the development of a healthy immune system and the prevention of colon cancer, these are important findings in relation to potential probiotic properties of the strain. The strain mainly affected saccharolytic fermentation, without influencing strongly proteolytic fermentation.

HU58 induced specific changes in the gut microbiota composition as indicated by specific changes in the DGGE community fingerprints.

Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments, the values may range in value above or below the stated value in a range of approximately ±5%; in other embodiments, the values may range in value above or below the stated value in a range of approximately ±2%; in other embodiments, the values may range in value above or below the stated value in a range of approximately ±1%. The preceding ranges are intended to be made clear by context, and no further limitation is implied.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entireties for all purposes.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

The present invention may be embodied in other specific forms without departing from the spirit or essential attributes thereof, and, accordingly, reference should be made to the appended claims, rather than to the foregoing specification, as indicating the scope of the invention.

We claim:

1. A method for modulating microbial metabolic activity or microbial community composition in a human subject, comprising administering to the human subject an effective amount of a spore-based probiotic composition comprising strains Bacillus indicus (HU36), Bacillus subtilis (HU58), Bacillus coagulans SC-208, Bacillus clausii SC-109, and Bacillus licheniformis SL-307, each strain comprising Bacillus spores, wherein a health outcome is improved in the human subject.

2. The method of claim 1, wherein the health outcome is protection against a condition selected from the group consisting of obesity-related disorders, metabolic disorders, inflammation, and cancer.

3. The method of claim 1, wherein one or more of acetate, propionate, or butyrate is increased in the human gastro-intestinal tract

4. The method of claim 1, wherein the spore-based probiotic composition is administered in a daily dose of about 4×10⁹ Bacillus spores (CFUs).

5. A method for increasing microbial diversity in the gastro-intestinal tract in a human subject, comprising administering to the human subject an effective amount of a spore-based probiotic composition comprising strains Bacillus indicus (HU36), Bacillus subtilis (HU58), Bacillus coagulans SC-208, Bacillus clausii SC-109, and Bacillus licheniformis SL-307, each strain comprising Bacillus spores.

6. The method of claim 5, wherein Bifidobacteriaceae family colonization is increased in the human gastro-intestinal tract.

7. The method of claim 5, wherein Faecalibacterium prausnitzii colonization is increased in the human gastro-intestinal tract.

8. The method of claim 5, wherein Akkermansia muciniphila colonization is increased in the human gastro-intestinal tract.

9. The method of claim 5, wherein the Firmicutes:Bacteroidetes phyla ratio is increased by about 50% or greater in the human gastro-intestinal tract.

10. The method of claim 5, wherein the spore-based probiotic composition is administered in a daily dose of about 4×10⁹ Bacillus spores (CFUs).
